SEQUENCE LISTING (30 sequences)

SEQ ID NO: 1 - DNA, 582 bp, Homo sapiens
  hEPOwt
atgggggtgcacgaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacgacgggctgtgctgaacactgcagcttgaatgagaatatcactgtcccagacaccaaagttaatttctatgcctggaagaggatggaggtcgggcagcaggccgtagaagtctggcagggcctggccctgctgtcggaagctgtcctgcggggccaggccctgttggtcaactcttcccagccgtgggagcccctgcagctgcatgtggataaagccgtcagtggccttcgcagcctcaccactctgcttcgggctctgggagcccagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacagatga

SEQ ID NO: 2 - DNA, 495 bp, Homo sapiens
  hs3
atgggggtgcacgaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacggtcgggcagcaggccgtagaagtctggcagggcctggccctgctgtcggaagctgtcctgcggggccaggccctgttggtcaactcttcccagccgtgggagcccctgcagctgcatgtggataaagccgtcagtggccttcgcagcctcaccactctgcttcgggctctgggagcccagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacagatga

SEQ ID NO: 3 - DNA, 489 bp, Homo sapiens
  hs4
atgggggtgcacgaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacgacgggctgtgctgaacactgcagcttgaatgagaatatcactgtcccagacaccaaagttaatttctatgcctggaagaggatggagccgtgggagcccctgcagctgcatgtggataaagccgtcagtggccttcgcagcctcaccactctgcttcgggctctgggagcccagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacagatga

SEQ ID NO: 4 - DNA, 525 bp, Homo sapiens
  h1-4
atgggggtgcacgaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacgacgggctgtgctgaacactgcagcttgaatgagaatatcactgtcccagacaccaaagttaatttctatgcctggaagaggatggaggtcgggcagcaggccctgttggtcaactcttcccagccgtgggagcccctgcagctgcatgtggataaagccgtcagtggccttcgcagcctcaccactctgcttcgggctctgggagcccagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacagatga

SEQ ID NO: 5 - DNA, 495 bp, Homo sapiens
  h1-5
atgggggtgcacgaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacgacgggctgtgctgaacactgcagcttgaatgagaatatcactgtcccagacaccaaagttaatttctatgccctgttggtcaactcttcccagccgtgggagcccctgcagctgcatgtggataaagccgtcagtggccttcgcagcctcaccactctgcttcgggctctgggagcccagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacagatga

SEQ ID NO: 6 - DNA, 475 bp, Homo sapiens
  h1-1
atgggggtgcacgaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacgacgggctgtgctgaacactgcagcttgaatgagaatatcactgtcccaggccctgttggtcaactcttcccagccgtgggagcccctgcagctgcatgtggataaagccgtcagtggccttcgcagcctcaccactctgcttcgggctctgggagcccagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacagatga

SEQ ID NO: 7 - DNA, 301 bp, Homo sapiens
  h2-1
atgggggtgcacgaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacgacgggctgtgctgaacactgcagcttgaatgagaacaatcactgctgacactttccgcaaactcttccgagtctactccaatttcctccggggaaagctgaagctgtacacaggggaggcctgcaggacaggggacagatga

SEQ ID NO: 8 - protein, 193 aa, Homo sapiens
  hEPOwt
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 9 - protein, 164 aa, Homo sapiens
  EV-3
MGVHECPAWLWLLLSLLSLPLGLPVLGAPPRLICDSRVLERYLLEAKEAENITVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 10 - protein, 162 aa, Homo sapiens
  EV-4
MGVHECPAWLWLLLSLLSLPLGLPVLGAPPRLICDSRVLERYLLEAKEAENITTGCAEHCSLNENITVPDTKVNFYAWKRMEPWEPLQLHVDKAVSGLRSLTTLLRALGAQKEAISPPDAASAAPLRTITADTFRKLFRVYSNFLRGKLKLYTGEACRTGDR

SEQ ID NO: 11 - protein, 174 aa, Homo sapiens
  EV1-4
MGVHECPAWLWLLLSLLSLPLGLPVLGAPPRLICDSRVLERYLLEAKEAENITTGCAEHCSLNENITVPDTKVNFYAWKRMEVGQQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 12 - protein, 164 aa, Homo sapiens
  EV1-5
MGVHECPAWLWLLLSLLSLPLGLPVLGAPPRLICDSRVLERYLLEAKEAENITTGCAEHCSLNENITVPDTKVNFYALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 13 - protein, 87 aa, Homo sapiens
  EV1-1
MGVHECPAWLWLLLSLLSLPLGLPVLGAPPRLICDSRVLERYLLEAKEAENITTGCAEHCSLNENITVPPGVGQLFPAVGAPAAACG

SEQ ID NO: 14 - protein, 68 aa, Homo sapiens
  EV2-1
MGVHECPAWLWLLLSLLSLPLGLPVLGAPPRLICDSRVLERYLLEAKEAENITTGCAEHCSLNENNHC

SEQ ID NO: 15 - protein, 10 aa, Artificial Sequence
  Synthesized
DITVGQQAVE

SEQ ID NO: 16 - protein, 8 aa, Artificial Sequence
  Synthesized
DITTGCAE

SEQ ID NO: 17 - protein, 137 aa, Artificial Sequence
  EV-3 aa 28-164
APPRLICDSRVLERYLLEAKEAENITVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 18 - protein, 135 aa, Artificial Sequence
  EV-4 aa 28-162
APPRLICDSRVLERYLLEAKEAENITTGCAEHCSLNENITVPDTKVNFYAWKRMEPWEPLQLHVDKAVSGLRSLTTLLRALGAQKEAISPPDAASAAPLRTITADTFRKLFRVYSNFLRGKLKLYTGEACRTGDR

SEQ ID NO: 19 - DNA, 20 bp, Artificial Sequence
  Spec EPO Fw primer
gtgctgaacactgcagcttg

SEQ ID NO: 20 - DNA, 20 bp, Artificial Sequence
  Spec EPO rev primer
cagacttctacggcctgctg

SEQ ID NO: 21 - DNA, 20 bp, Artificial Sequence
  Spec hs3 fwd primer
ccgagaatatcacggtcggg

SEQ ID NO: 22 - DNA, 20 bp, Artificial Sequence
  Spec hs3 rev
cggctttatccacatgcagc

SEQ ID NO: 23 - DNA, 19 bp, Artificial Sequence
  Spec hs4 fwd primer
gaagaggatggagccgtgg

SEQ ID NO: 24 - DNA, 20 bp, Artificial Sequence
  Spec hs4 rev
atctggaggggagatggctt

SEQ ID NO: 25 - DNA, 20 bp, Artificial Sequence
  EPOR fwd primer
atcctgacgctctccctcat

SEQ ID NO: 26 - DNA, 20 bp, Artificial Sequence
  EPOR rev primer
aggccttcaaactcgctctc

SEQ ID NO: 27 - DNA, 17 bp, Artificial Sequence
  EPHB4 fwd primer
gtcccgcgcggagtatc

SEQ ID NO: 28 - DNA, 20 bp, Artificial Sequence
  EPHB4 rev primer
cttctcctgacaggggcttg

SEQ ID NO: 29 - DNA, 20 bp, Artificial Sequence
  CSF2RB fwd primer
agtgggaggagaagatcccc

SEQ ID NO: 30 - DNA, 20 bp, Artificial Sequence
  CSF2RB rev primer
cccaggatgtcaggtaggga

(19) United States
(12) Patent Application Publication
(10) Pub. No.: US 2025/0333489 A1
(43) Pub. Date: Oct. 30, 2025

(54) EPO VARIANTS AND MODULATORS

(71) Applicant: ANDREMACON S.R.L., Milano (IT)

(72) Inventors: Giovanni Marfia, Milano (IT); Stefania Elena Navone, Milano (IT); Giuseppe Scalvini, Castelletto di Leno (IT); Rolando Campanella, Milano (IT); Giovanni Andrea Alotta, Altofonte (PA) (IT); Emanuela Barilla, Parma (IT)

(73) Assignee: ANDREMACON S.R.L., Milano (IT)

(21) Appl. No.: 18/569,499

(22) PCT Filed: Jun. 15, 2021
(86) PCT No.: PCT/IB2021/055266

§ 371 Date: Dec. 12, 2023

(51) Int. Cl. C07K 16/22; A61K 39/00; A61P 35/00; C12Q 1/6886

(52) U.S. Cl. CPC C07K 16/22; A61P 35/00; C12Q 1/6886; A61K 2039/505; C12Q 2600/158

(57) ABSTRACT

The invention relates to negative functional modulators of at least one variant of the non-erythrogenic erythropoietin (V-EPO) and pharmaceutical compositions or kits containing them. Such functional negative modulators of V-EPO may be a mono- or multi-specific antibody anti-EV3, anti-EV4, anti-EV1-4, anti-EV1-5, anti-EV1-1, or EV2-1, or anti-Epo receptor (EpoR) anti-EPHB4, anti-CSF2RB, an antisense oligonucleotide, DNA decoy, RNA decoy, a ribozyme, an antagomiR, a shRNA, LNA or siRNA.

Several uses of these functional modulators are described, which have been advantageously employed as a medicament and for the treatment of an oncological pathology, a proliferative pathology, chronic inflammatory diseases on an autoimmune and non-autoimmune basis, of neurodegenerative diseases, and in the treatment of patients undergoing organ or tissue transplantation.

The invention also describes variants of EPO for use in the diagnosis and in the treatment of an oncological pathology, a proliferative pathology, chronic inflammatory diseases on an autoimmune and non-autoimmune basis, of neurodegenerative diseases, and in the treatment of patients undergoing an organ or tissue transplantation and as a diagnostic agent.

According to another aspect, a monoclonal antibody to at least one of the variants of the erythropoietin is described.

According to yet another aspect, the use of at least one alternative splicing variant of non-erythrogenic EPO and the measurement thereof at tissue and/or systemic level is described, as well as the study of the methylation status of the promoters of the genes involved in the EPO signalling pathway (by way of example EPO, EPOR, EPHB4, CSF2RB), as diagnostic, prognostic and predictive markers of an oncological pathology, a proliferative pathology, neurodegenerative or inflammatory pathology.

Sequence listing ASCII file P022503US-01_sequence listing_240604.txt created on Jun. 4, 2024 and of 19,401 bytes is incorporated herein by reference.

### FIELD OF THE INVENTION

The invention relates to negative functional modulators of at least one variant of the non-erythrogenic erythropoietin (V-EPO) and pharmaceutical compositions or kits containing them. Such functional negative modulators of V-EPO may be a mono- or multi-specific antibody anti-EV3, anti-EV4, anti-EV1-4, anti-EV1-5, anti-EV1-1, or EV2-1, or anti-Epo receptor (EpoR) anti-EPHB4, anti-CSF2RB, an antisense oligonucleotide, DNA decoy, RNA decoy, a ribozyme, an antagomiR, a shRNA, LNA or siRNA.

Several uses of these functional modulators are described, which have been advantageously employed as a medicament and for the treatment of an oncological pathology, a proliferative pathology, chronic inflammatory diseases on an autoimmune and non-autoimmune basis, of neurodegenerative diseases, and in the treatment of patients undergoing organ or tissue transplantation.

The invention also describes variants of EPO for use in the treatment of an oncological pathology, a proliferative pathology, chronic inflammatory diseases on an autoimmune and non-autoimmune basis, of neurodegenerative diseases, and in the treatment of patients undergoing an organ or tissue transplantation and as a diagnostic agent. According to another aspect, a monoclonal antibody to at least one of the variants of the erythropoietin is described.

### STATE OF THE ART

Monoclonal antibodies (mABs) are the fastest growing market segment in the pharmaceutical industry. The mABs are particularly appreciated among biotherapists for their unique characteristics, such as high target specificity, favourable pharmacokinetics (high half-life), fast development and high success rate compared to small molecules. Today, millions of people are suffering from cancer or have had cancer. Neoplasms are a group of diseases characterised by uncontrolled growth, invasiveness and spread of abnormal cells. The involvement of stem and endothelial components in the formation and development of the neoplasm is now evident. Indeed, experimental evidence has shown that cancer stem cells (CSCs) guide the growth of the tumour hierarchically, also through bidirectional communication with the vascular compartment, influencing the microenvironment and the response to chemo- and radiotherapy. CSCs are responsible for the processes of tumour initiation and maintenance and also for tumour resistance to therapeutic treatment and, consequently, the occurrence of relapses. As such, CSCs are an important therapeutic target, but the mechanisms underlying their pathobiology are still poorly understood, making it difficult to identify molecules capable of targeting them.

Human erythropoietin (Epo) is a 30.4 kDa glycoprotein produced and secreted mainly by the kidneys. Epo is normally present in the circulatory stream where it is the main erythropoietic hormone. Through its binding to a surface receptor, EpoR, Epo is responsible for regulating red blood cell production by stimulating the differentiation and proliferation of erythroid progenitors, as well as for maintaining the erythroid series itself. The gene encoding EPO is located on chromosome 7 (7q11-22) and consists of 5 exons and 4 introns that transcribe for a single polypeptide of 193 amino acids. This polypeptide undergoes specific post-translational modifications consisting of glycosylation, formations of di-sulphur bridges and removal of the hydrophobic sequence of 27 amino acids, known as the leader sequence. Epo synthesis is controlled by a very sensitive feedback system whose production and secretion depends on the alterations in the oxygen supply. In fact, its synthesis mechanism is based on the presence of the transcription factor Hypoxia Inducible Factor (HIF). In parallel, hypoxia also plays a key role in controlling tumour growth and angiogenesis and is an effective mechanism of tumour adaptation and survival. The genes involved in the hypoxia signalling pathway are in fact overexpressed by CSCs in the hypoxic vascular/perinecrotic niche, but not by the transitional tissue that is present at the resection margin, which is considered “disease-free” in anatomo-pathological terms. The use of Epo and the derivatives thereof is well known in the treatment of anaemia due to renal failure, reduced erythropoiesis and in combination with myelosuppressive chemotherapy regimens in the treatment of neoplasms, although the treatment with Epo involves reaching blood concentrations very far from the physiological ones. Recently, transcriptional variants of EPO (V-EPO), due to alternative splicing mechanisms, which are physiologically present in the circulatory stream with a non-erythropoietic function, have been described.

Alternative splicing plays an important role in the evolution of eukaryotic organisms. Recent studies have shown that approximately 40-60% of human genes undergo an alternative splicing event, resulting in a wide range of protein isoforms.

In the case of V-EPO, the alternative splicing mechanisms leading to the loss of exon 3 or exon 4 (defined as hs3 and hs4, respectively) translate proteins that turn out to be different from Epo. Epo and the EV-3 protein encoded by hs3 differ in 29 amino acids, with a consequent decrease in molecular mass from 20 kDa for Epo to 16.8 kDa for EV-3.

In the glycosylated form of the two proteins, the loss of an N-glycosylation site (Asn-38) in EV-3 leads to a sharper difference in apparent molecular weight (˜5-8 kDa). The human protein Epo has a four-helix bundle structure, which is typical of haematopoietic growth factors. The four long α-helices (A, B, C and D) are connected by two long cross-rings (AB and CD) and a short ring (BC). Near the carboxy-terminal end of the AB ring there is a short alpha-helical segment (mini-helix B′) that is important for binding to the EpoR receptor. As described above, the sequence of the EV-3 protein suggests the loss of the AB ring, which results in a considerable change in the tertiary structure compared to Epo. The systematic processing of the Epo and EV-3 sequences has enabled the generation of two peptides that allow the identification and differentiation of Epo and EV-3 within a sample. The resulting peptide with the sequence “DITVGQQAVE” (SEQ ID NO:15) is contained in EV-3 but not in Epo and no other protein including that sequence was identified. Therefore, this peptide is considered to be the specific identifier of EV-3. On the other hand, the peptide sequence “DITTGCAE” (SEQ ID NO: 16) is absent in EV-3 and is only contained in human Epo and therefore it serves as an Epo-specific identifier.

Experiments carried out on CD34+ human haematopoietic progenitors have shown that EV-3 administration, unlike Epo, does not stimulate the formation of erythropoietic colonies (CFU-E). Since the CD34+ haematopoietic stem cells express EpoR, the absence of the stimulating effect of EV-3 might be due to the absence of the EpoR binding site in the EV-3 splicing variant.

This observation is consistent with the loss of the AB ring in the EV-3 protein sequence, which is present in the Epo protein. The AB ring contains a segment that has been identified as important for binding to EpoR and formation of the homodimer complex. Surprisingly, our results show that brain tumour cells express significantly increased levels of the natural variants of EPO, hs3 and hs4, compared to healthy brain cells and that there is a considerable and statistically significant enrichment of EPO, hs3 and hs4 in primary cancer stem and endothelial cells compared to differentiated tumour cells composing the tumour mass. The loss of the binding site with the EpoR receptor, which is responsible for triggering the erythropoietic mechanism, results in the disappearance of the erythropoietic activity of hs3 and hs4, which however retain the ability to induce cell proliferation through binding to alternative receptors.

As mentioned above, Epo's signalling pathway is mediated by its binding to a surface receptor (EpoR), a transmembrane glycoprotein (PM: 66-78 kDa) belonging to the cytokine receptor superfamily, mainly located on progenitors present in the bone marrow.

Expression of EpoR in non-haematopoietic cells such as vascular endothelial cells, shows in the kidney, myoblasts and intestine that there are non-haematopoietic biological effects of Epo-EpoR signalling. In particular, recent studies have reported the expression of EpoR in tissue biopsies of breast cancer, ovarian and uterine cancers, melanoma and renal carcinoma, suggesting a mechanism of induction of uncontrolled cell proliferation mediated by the Epo-EpoR binding.

There are two forms of EpoR: a homodimeric form, responsible for the erythropoietic effects, and a heterodimeric form, consisting of an EpoR chain and a β-common receptor chain (βcR, CD131, colony-stimulating factor 2 receptor-β, CSF2RB). This second receptor is responsible for the non-erythropoietic effects of Epo in several organs including the heart, nervous system, intestine, uterus, kidney and pancreatic islets. The activation of the EpoR/CD131 heterodimer requires much higher concentrations of Epo than those required for the activation of the homodimeric EpoR and results in the transduction of signals that are partly shared with the homodimeric EpoR. In particular, both induce the activation of PI3K and MAPK, the phosphorylation of STAT5 and the regulation of the binding activity of NF-kB family members.

The presence of a third receptor, called ephrin-type B receptor 4 (EphB4), which differs from other Eph receptors in the presence of an isoleucine instead of a tyrosine at position 48 in the hydrophobic cavity, has also been demonstrated. EphB4 has been shown to interact predominantly with Ephrin B2 but is also able to act as a functional Epo receptor. Studies carried out on ovarian carcinoma cells, which constitutively express both EphB4 and EpoR, have shown that both ephrin-B2 and Epo directly activate EphB4, causing an increased proliferation and invasive migration mediated by the kinase Scr, and STAT3. In contrast, the activation of EpoR in the same ovarian carcinoma cells resulted in the activation of the JAK/STAT signalling pathway. The direct activation of EphB4 by Epo was further confirmed in COS-1 cells transfected with EphB4 that did not express EpoR endogenously. Both Ephrin-B2 and Epo independently activate EphB4 and may act synergistically when released on the same target cells. Binding studies showed a low binding affinity of Epo for EphB4 (KD of 880 nM), compared to a KD of 28 nM for EpoR. In addition, in a clinical trial it was observed that the survival of breast cancer patients was significantly reduced with high expression levels of EphB4, but not of EpoR in the tumour cells, and that the treatment with Epo decreased survival more. This indicated that Epo supported tumour growth, particularly by activating the mechanisms initiated by EphB4.

Surprisingly, our results show that glioma cancer stem cells exhibit a constitutive overexpression of EPHB4 and CSF2RB, which coincides with a significant decrease in EPOR expression. These data suggest that Epo and Ev-3 perform their functions by acting selectively on different receptors: through binding to EpoR, Epo activates the signal pathway linked to erythropoiesis in haematopoietic cells; conversely, both Epo and EV-3, through binding to the EPHB4 and CSF2RB receptors, activate the pathological process linked to the proliferation, angiogenesis and survival of tumour cells, with autocrine and paracrine mechanisms thanks to which they promote the progression of neoplastic disease.

Our previous researches show that Epo functions as a growth factor for glioblastoma tumour cells and that blocking the signalling pathway by means of a monoclonal antibody is able to inhibit the growth of both the stem and endothelial component, to induce apoptosis, and to decrease the functionality of endothelial cells by inhibiting the formation of vascular structures and migration (WO/2015/189813).

In this respect, the present invention demonstrates that, while in healthy cells, the transcriptional variants of EPO (V-EPO) are produced in a small manner and act only in response to environmental stimuli such as hypoxia, in tumour cells, and particularly in CSCs, their expression is stable and constitutive. CSCs show an unbalanced genotypic and phenotypic rearrangement in favour of gene duplication of EPO and the receptors thereof, as well as a constitutive expression of EPO splicing variants such as hs3 and hs4, which play a key role in cell survival and therapy resistance. From the studies carried out to demonstrate the present invention it emerges that in contrast to healthy cells, CSCs show at the same time an increased expression of the hs3 and hs4 variants of EPO, CSF2RB and EPHB4, specific receptors of the neoplastic process, to which a reduced expression of EpoR is associated, which is responsible for the haematopoietic process.

The discovery of a specific constitutive expression pattern of the variants of EPO and the receptors thereof that goes beyond the physiological role of Epo linked to erythropoiesis, demonstrated in the present invention, thus allows the identification of new easily analysable diagnostic, prognostic and predictive markers such as hs3, hs4, EPHB4 and CSF2RB that can also predict the prognosis of the patients and the response to therapy.

Moreover, given the lack of a binding site to the EpoR receptor that mediates the erythropoietic cascade, V-EPOs, whose expression is significantly increased, lose their erythropoietic function but retain the biological oncopromoting functions of the wild type protein by binding to alternative receptors such as EPHB4 and CSF2RB, which are also significantly overexpressed in oncological models.

From these premises, it is clear that the specific blockade of the transcriptional variants not associated with haematopoiesis and of the receptors that mediate the intracellular signalling could constitute a treatment with important benefits for the patients suffering from oncological pathologies and associated clinical manifestations, while minimising the impairment of the erythrogenic function.

Therefore, the present invention takes into consideration the co-formulations comprising functional modulators of the transcriptional variants of EPO and/or the above indicated receptors (EpoR, EPHB4, CSF2RB), the effect of which envisages to specifically and bidirectionally block Epo's oncopromoting signalling pathway, excluding the involvement of its erythropoietic activity. Such simultaneous blockade can be actuated through co-administered single antibodies, or specially constructed antibodies in which the Fab′ and scFv fragments of the antibodies can be constructed by attaching many fragments to the surface of carriers, like in the immunopolysomes, or by producing, by chemical conjugation or genetic engineering, bivalent (diabodies, 60 kDa), trivalent (triabodies, 90 kDa) or tetravalent (tetrabodies, 120 kDa) fragments, thereby obtaining multivalent constructs with higher binding avidity. Alternatively, other antibody formats consisting of scFv may be used, such as by way of example the bispecific/trispecific diabodies, which are formed by two/three scFv fragments, one of which is directed against a tumour molecule and the other one against a surface molecule present on the cytotoxic effector cell and/or against a circulating molecule, such as the EPO variant.

The data of the present invention demonstrate that the constitutive expression of V-EPO in CSCs induces an increased synthesis and release of sphingosine-1-phosphate (S1P), which results in a specific synergistic mechanism of the CSCs and influences the response thereof to chemo- and radiotherapy.

S1P, which represents the most potent oncopromoting protein lipid, exerts its action by stimulating proliferation, survival and drug resistance of tumour cells and cancer stem cells, as well as by promoting the angiogenesis by acting on the stem and endothelial cells through binding to specific G-protein-coupled receptors, originally known as EDGs and now called S1P receptors (S1PRs). Glioblastoma cells, and in particular glioblastoma stem cells (GSCs), have been demonstrated to synthesise S1P and export it to the extracellular environment where it promotes resistance to chemotherapeutics. Marfia et al., 2014 demonstrated that highly proliferating glioblastoma cancer stem cells are able to synthesise and release 10-fold higher levels of S1P into the extracellular compartment than slowly growing cells (Marfia et al., 2014 Glia. 62(12):1968-81. DOI:10.1002/glia.22718. PMID: 25042636). The same research group demonstrated that glioblastoma endothelial cells (GECs) also synthesise and release S1P into the extracellular environment, and when cultured in co-culture with cancer stem cells, they significantly increase the expression of a key enzyme for S1P synthesis, kinase 2 (Sphk2), with a further significant increase in S1P production. High levels of S1P through an autocrine and paracrine system stimulate the growth of CSCs and the migration of GECs, exacerbating tumour mass growth (Abdel Hadi L et al. 2018 Biochim Biophys Acta Mol Cell Biol Lipids. 1863(10):1179-1192. DOI:10.1016/j.bbalip.2018.07.009. PMID: 30056170). Because of its important role in the control of cell proliferation and viability, the interaction of S1P with its receptors, already studied for the impact thereof in autoimmune diseases, is the subject of intensive studies, with the aim of identifying new and more effective anticancer drugs. To date, FTY720, a sphingosine analogue which, after phosphorylation, acts as a functional antagonist of S1PRs, has been identified. FTY720 has been turned out to be effective in post-organ transplantation therapy, in multiple sclerosis and, in vitro, in decreasing tumour growth in different types of tumours. Numerous efforts in basic, pre-clinical and clinical research are made to identify new effective treatments. There is a strong need for new therapeutic approaches to treat tumours that are also effective at the level of cancer stem cells and also in particularly aggressive solid tumours, such as glioblastoma, breast, colorectal, lung, prostate, uterine and ovarian cancer, and in non-solid tumours, such as leukaemia in general and in particular chronic myeloid leukaemia.

Therefore, it is a further object of the present invention to provide products and compositions including the co-administration of inhibitors of the Epo variants and/or the receptors thereof and inhibitors of the sphingolipid signalling pathway, for the treatment of an oncological pathology, a proliferative pathology, chronic inflammatory diseases on an autoimmune or non-autoimmune basis, and in the treatment of patients undergoing organ or tissue transplantation.

### SUMMARY OF THE INVENTION

The invention concerns functional negative modulators of the biological and/or synthetic variants of erythropoietin (V-EPO) and the pharmaceutical compositions or kits containing them. Specifically, a negative functional modulator of at least one natural or synthetic variant of EPO is described, wherein said variant is selected from the group consisting of: EV3 variant having the amino acid sequence SEQ ID NO: 9, EV4 variant having the amino acid sequence SEQ ID NO: 10, EV1-4 variant having the amino acid sequence SEQ ID NO: 11, EV1-5 variant having the amino acid sequence SEQ ID NO: 12, EV1-1 variant having the amino acid sequence SEQ ID NO: 13, EV2-1 variant having the amino acid sequence SEQ ID NO: 14, wherein said negative functional modulator is a mono- or multi-specific antibody anti-EV3, anti-EV4, anti-EV1-4, anti-EV1-5, anti EV1-1, or EV2-1, and/or anti-Epo receptor (EpoR, EPHB4, CSFR2B), an antisense oligonucleotide, DNA decoy, RNA decoy, a ribozyme, an antagomiR, a shRNA, LNA or siRNA. In a preferred embodiment, said antibody is a monoclonal antibody.

According to a second aspect, the present invention concerns a negative functional modulator of at least one variant of EPO, wherein said variant is selected from the group consisting of: EV3 variant having the amino acid sequence SEQ ID NO: 9, EV4 variant having the amino acid sequence SEQ ID NO: 10, EV1-4 variant having the amino acid sequence SEQ ID NO: 11, EV1-5 variant having the amino acid sequence SEQ ID NO: 12, EV1-1 variant having the amino acid sequence SEQ ID NO: 13, EV2-1 variant having the amino acid sequence SEQ ID NO: 14, for use as a medicament.

According to a third aspect, the present invention concerns a functional negative modulator of at least one V-EPO, wherein said variant is selected from the group consisting of: EV3 variant having the amino acid sequence SEQ ID NO: 9, EV4 variant having the amino acid sequence SEQ ID NO: 10, EV1-4 variant having the amino acid sequence SEQ ID NO: 11, EV1-5 variant having the amino acid sequence SEQ ID NO: 12, EV1-1 variant having the amino acid sequence SEQ ID NO: 13, EV2-1 variant having the amino acid sequence SEQ ID NO: 14, for use in the treatment of an oncological pathology, a proliferative pathology, in the therapy of chronic inflammatory diseases on an autoimmune and non-autoimmune basis, of neurodegenerative diseases, and in the treatment of patients undergoing organ or tissue transplantation.

According to a fourth aspect, a variant of the erythropoietin (V-EPO) is described which is selected from the group consisting of: EV3 variant with the amino acid sequence SEQ ID NO: 9, EV4 variant having the amino acid sequence SEQ ID NO: 10, EV1-4 variant having the amino acid sequence SEQ ID NO: 11, EV1-5 variant having the amino acid sequence SEQ ID NO: 12, EV1-1 variant having the amino acid sequence SEQ ID NO: 13, EV2-1 variant having the amino acid sequence SEQ ID NO: 14, for use as a diagnostic agent.

According to a fifth aspect, a pharmaceutical composition is described comprising a negative functional modulator of at least one variant of the erythropoietin (EPO) selected from the group consisting of: EV3 variant having the amino acid sequence SEQ ID NO: 9, EV4 variant having the amino acid sequence SEQ ID NO: 10, EV1-4 variant having the amino acid sequence SEQ ID NO: 11, EV1-5 variant having the amino acid sequence SEQ ID NO: 12, EV1-1 variant having the amino acid sequence SEQ ID NO: 13, EV2-1 variant having the amino acid sequence SEQ ID NO: 14, and/or anti Epo receptor (EpoR, EPHB4, CSF2RB), an antisense oligonucleotide, DNA decoy, RNA decoy, a ribozyme, an antagomiR, a shRNA, LNA or siRNA, for use in the treatment of an oncological pathology, a proliferative pathology, chronic inflammatory diseases on an autoimmune and non-autoimmune basis, and in the treatment of patients undergoing organ or tissue transplantation.

According to a sixth aspect, a pharmaceutical kit is described comprising two or more components selected from the group consisting of:
A negative functional modulator of at least one variant of the erythropoietin (EPO), wherein said variant is selected from the group consisting of: EV3 variant having the amino acid sequence SEQ ID NO: 9, EV4 variant having the amino acid sequence SEQ ID NO: 10, EV1-4 variant having the amino acid sequence SEQ ID NO: 11, EV1-5 variant having the amino acid sequence SEQ ID NO: 12, EV1-1 variant having the amino acid sequence SEQ ID NO: 13, EV2-1 variant having the amino acid sequence SEQ ID NO: 14;
a monoclonal antibody to at least one of the at least one variant of the erythropoietin (EPO), wherein said variant is selected from the group consisting of: EV3 variant having the amino acid sequence SEQ ID NO: 9, EV4 variant having the amino acid sequence SEQ ID NO: 10, EV1-4 variant having the amino acid sequence SEQ ID NO: 11, EV1-5 variant having the amino acid sequence SEQ ID NO: 12, EV1-1 variant having the amino acid sequence SEQ ID NO: 13, EV2-1 variant having the amino acid sequence SEQ ID NO: 14, wherein said antibody is a bispecific antibody, diabody, tryabody, or tetrabody;
a polynucleotide encoding a variant of the erythropoietin (EPO), wherein said variant is selected from the group consisting of: EV3 variant having the amino acid sequence SEQ ID NO: 9, EV4 variant having the amino acid sequence SEQ ID NO: 10, EV1-4 variant having the amino acid sequence SEQ ID NO: 11, EV1-5 variant having the amino acid sequence SEQ ID NO: 12, EV1-1 variant having the amino acid sequence SEQ ID NO: 13, EV2-1 variant having the amino acid sequence SEQ ID NO: 14;
a chemotherapy drug;
a negative functional modulator of a natural or synthetic variant of the Epo receptors (EpoR, EPHB4, CSF2RB);
a negative functional modulator of the Epo receptors (EpoR EPHB4, CSF2RB);
a polynucleotide encoding Epo receptors (EpoR EPHB4, CSF2RB);
a negative functional modulator of the sphingosine-1-phosphate (S1P) signalling pathway;
an inhibitor of S1P and the metabolism thereof; or
mimetics of the EPO variants that preserve the erythropoietic function.

According to a seventh aspect, a monoclonal antibody to at least one of the variants of the erythropoietin (EPO) is described, wherein said variant is selected from the group consisting of: EV3 variant having the amino acid sequence SEQ ID NO: 9, EV4 variant having the amino acid sequence SEQ ID NO: 10, EV1-4 variant having the amino acid sequence SEQ ID NO: 11, EV1-5 variant having the amino acid sequence SEQ ID NO: 12, EV1-1 variant having the amino acid sequence SEQ ID NO: 13, EV2-1 variant having the amino acid sequence SEQ ID NO: 14.

According to an eighth aspect, a negative functional modulator of at least one variant of the erythropoietin (EPO) is described, wherein said variant is selected from the group consisting of: EV3 variant having the amino acid sequence SEQ ID NO: 9, EV4 variant having the amino acid sequence SEQ ID NO: 10, EV1-4 variant having the amino acid sequence SEQ ID NO: 11, EV1-5 variant having the amino acid sequence SEQ ID NO: 12, EV1-1 variant having the amino acid sequence SEQ ID NO: 13, EV2-1 variant having the amino acid sequence SEQ ID NO: 14, for use as a diagnostic agent.

Dependent claims describe particular embodiments of the invention.

### DESCRIPTION OF THE DRAWINGS

The invention will now be described in detail and with reference to the following accompanying Figures.

FIG. 1. Genetic analysis of copy number variations that are present in glioblastoma tissue and correlation with histological diagnosis and survival of brain tumour patients. In FIG. 1A: the EPHB1 and EPHB3 genes, in FIG. 1B the EPO, EPHA1, EPHB4 and EPHB6 genes and in FIG. 1C EPOR.

FIG. 2. Gene expression of EPO, hs3 and hs4 cancer stem and endothelial cells isolated from the brain tissue (FIGS. 2A and 2C) and tumour cell lines (FIG. 2B) and in relation to survival (FIG. 2D).

FIG. 3. Gene expression of EpoR, EPHB4, CSF2RB in cancer stem and endothelial cells isolated from the brain tissue (FIGS. 3A and 3C) and tumour cell lines (FIG. 3C) and in relation to survival (FIG. 3D).

FIG. 4. Viability analysis on tumour cells after treatment with anti-EV3: FIG. 4A Primary GSCs, FIG. 4B Primary GECs, FIG. 4C Primary Anaplastic Astrocytoma Cells, FIG. 4D DLD1, FIG. 4E PC-3 and FIG. 4F LNCAP.

FIG. 5. Viability analysis on tumour cells after treatment with anti-EV3: FIG. 5A MCF-7, FIG. 5B K562, FIG. 5C A2780, FIG. 5D A549, FIG. 5E SHSY-5Y and FIG. 5F Human astrocytes.

FIG. 6. Analysis of the migration of cancer endothelial cells isolated from brain biopsies after treatment with anti-EV3, wherein FIG. 6A shows the images acquired under the microscope and FIG. 6B the graph showing the values.

FIG. 7. Analysis of the migration of DLD1 tumour cells after treatment with anti-EV3 (FIG. 7A) and the graph showing the values (FIG. 7B).

FIG. 8. Analysis of the formation of tubular structures on cancer endothelial cells isolated from brain tumour biopsies after treatment with anti-EV3. FIG. 8A shows the microscope images and FIG. 8B the graph showing the values.

FIG. 9. Viability analysis on tumour cells after treatment with anti-EPOR, anti-EPHB4, anti-CSF2RB alone or in combination with anti-EV3 and/or FTY720: in FIG. 9A Primary GSCs, FIG. 9B DLD1, FIG. 9C PC3, FIG. 9D MCF-7, FIG. 9E A2780, FIG. 9F A549.

FIG. 10. Viability analysis on human astrocytes after treatment with anti-EPOR, anti-EPHB4, anti-CSF2RB alone or co-administered with anti-EV3 and/or FTY720.

### DETAILED DESCRIPTION OF THE INVENTION

The invention therefore concerns negative functional modulators of the biological and/or synthetic transcriptional variants of the erythropoietin (V-EPO). Specifically, a negative functional modulator of at least one V-EPO is described, wherein said variant is selected from the group consisting of: EV3 variant having the amino acid sequence SEQ ID NO: 9, EV4 variant having the amino acid sequence SEQ ID NO: 10, EV1-4 variant having the amino acid sequence SEQ ID NO: 11, EV1-5 variant having the amino acid sequence SEQ ID NO: 12, EV1-1 variant having the amino acid sequence SEQ ID NO: 13, EV2-1 variant having the amino acid sequence SEQ ID NO: 14, wherein said negative functional modulator is a mono- or multi-specific antibody anti-EV3, anti-EV4, anti-EV1-4, anti-EV1-5, anti EV1-1, or EV2-1, and/or anti-Epo receptor (EpoR, EPHB4, CSFR2B), an antisense oligonucleotide, DNA decoy, RNA decoy, a ribozyme, an antagomiR, a shRNA, LNA or siRNA.

Said functional EPO modulators can be identified for example by constituting phage display antibody libraries and hybridoma techniques and can be functional negative modulators of any recombinant portion or peptide having part of the primary structural conformation of human Epo or any natural variant or synthetic derivative possessing the biological and functional erythropoietic and/or non-erythropoietic property of Epo. Preferably, said modulators are peptides or peptide-mimetics that recognise and bind an epitope in AA 28-164 of EV-3: SEQ ID NO: 17 APPRLICDSRVLERYLLEAKEAENITVGQQAVEVWQGLALLSEAVLRGQALLVNSSQP WEPLQLHVDKAVSGLRSLTTLLRALGAQKEAISPPDAASAAPLRTITADTFRKLFRVY SNFLRGKLKLYTGEACRTGDR,

### and in AA 28-162 of EV-4: SEQ ID NO: 18 APPRLICDSRVLERYLLEAKEAENITTGCAEHCSLNENITVPDTKVNFYAWKRMEPWE PLQLHVDKAVSGLRSLTTLLRALGAQKEAISPPDAASAAPLRTITADTFRKLFRVYSNF LRGKLKLYTGEACRTGDR.

In the present invention:
“human (EPO) erythropoietin (HGNC: 3415; Entrez Gene: 2056; Ensembl: ENSG00000130427; OMIM: 133170)” means a nucleotide sequence of SEQ ID NO:1 ATGGGGGTGCACGAATGTCCTGCCTGGCTGTGGCTTCTCCTGTCCCTGCTGTCG CTCCCTCTGGGCCTCCCAGTCCTGGGCGCCCCACCACGCCTCATCTGTGACAGC CGAGTCCTGGAGAGGTACCTCTTGGAGGCCAAGGAGGCCGAGAATATCACGAC GGGCTGTGCTGAACACTGCAGCTTGAATGAGAATATCACTGTCCCAGACACCAAA GTTAATTTCTATGCCTGGAAGAGGATGGAGGTCGGGCAGCAGGCCGTAGAAGTC TGGCAGGGCCTGGCCCTGCTGTCGGAAGCTGTCCTGCGGGGCCAGGCCCTGTT GGTCAACTCTTCCCAGCCGTGGGAGCCCCTGCAGCTGCATGTGGATAAAGCCGT CAGTGGCCTTCGCAGCCTCACCACTCTGCTTCGGGCTCTGGGAGCCCAGAAGGA AGCCATCTCCCCTCCAGATGCGGCCTCAGCTGCTCCACTCCGAACAATCACTGC TGACACTTTCCGCAAACTCTTCCGAGTCTACTCCAATTTCCTCCGGGGAAAGCTG AAGCTGTACACAGGGGAGGCCTGCAGGACAGGGGACAGATGA, corresponding to the amino acid sequence of SEQ ID NO: 8 MGVHECPAWLWLLLSLLSLPLGLPVLGAPPRLICDSRVLERYLLEAKEAENITTGCAE HCSLNENITVPDTKVNFYAWKRMEVGQQAVEVWQGLALLSEAVLRGQALLVNSSQP WEPLQLHVDKAVSGLRSLTTLLRALGAQKEAISPPDAASAAPLRTITADTFRKLFRVY SNFLRGKLKLYTGEACRTGDR
“negative functional modulators of the variant of Erythropoietin (EPO) EV-3” means a molecule that binds the nucleotide sequence SEQ ID NO: 2 (called hs3) ATGGGGGTGCACGAATGTCCTGCCTGGCTGTGGCTTCTCCTGTCCCTGCTGTCG CTCCCTCTGGGCCTCCCAGTCCTGGGCGCCCCACCACGCCTCATCTGTGACAGC CGAGTCCTGGAGAGGTACCTCTTGGAGGCCAAGGAGGCCGAGAATATCACGGTC GGGCAGCAGGCCGTAGAAGTCTGGCAGGGCCTGGCCCTGCTGTCGGAAGCTGT CCTGCGGGGCCAGGCCCTGTTGGTCAACTCTTCCCAGCCGTGGGAGCCCCTGC AGCTGCATGTGGATAAAGCCGTCAGTGGCCTTCGCAGCCTCACCACTCTGCTTC GGGCTCTGGGAGCCCAGAAGGAAGCCATCTCCCCTCCAGATGCGGCCTCAGCT GCTCCACTCCGAACAATCACTGCTGACACTTTCCGCAAACTCTTCCGAGTCTACT CCAATTTCCTCCGGGGAAAGCTGAAGCTGTACACAGGGGAGGCCTGCAGGACAG GGGACAGATGA, having the amino acid sequence of SEQ ID NO: 9 MGVHECPAWLWLLLSLLSLPLGLPVLGAPPRLICDSRVLERYLLEAKEAENITVGQQA VEVWQGLALLSEAVLRGQALLVNSSQPWEPLQLHVDKAVSGLRSLTTLLRALGAQKE AISPPDAASAAPLRTITADTFRKLFRVYSNFLRGKLKLYTGEACRTGDR
“negative functional modulators of the variant of Erythropoietin (Epo) EV-4” means a molecule that binds the sequence SEQ ID NO: 3 (called hs4) ATGGGGGTGCACGAATGTCCTGCCTGGCTGTGGCTTCTCCTGTCCCTGCTGTCG CTCCCTCTGGGCCTCCCAGTCCTGGGCGCCCCACCACGCCTCATCTGTGACAGC CGAGTCCTGGAGAGGTACCTCTTGGAGGCCAAGGAGGCCGAGAATATCACGAC GGGCTGTGCTGAACACTGCAGCTTGAATGAGAATATCACTGTCCCAGACACCAAA GTTAATTTCTATGCCTGGAAGAGGATGGAGCCGTGGGAGCCCCTGCAGCTGCAT GTGGATAAAGCCGTCAGTGGCCTTCGCAGCCTCACCACTCTGCTTCGGGCTCTG GGAGCCCAGAAGGAAGCCATCTCCCCTCCAGATGCGGCCTCAGCTGCTCCACTC CGAACAATCACTGCTGACACTTTCCGCAAACTCTTCCGAGTCTACTCCAATTTCCT CCGGGGAAAGCTGAAGCTGTACACAGGGGAGGCCTGCAGGACAGGGGACAGAT GA, having the amino acid sequence of SEQ ID NO:10 MGVHECPAWLWLLLSLLSLPLGLPVLGAPPRLICDSRVLERYLLEAKEAENITTGCAE HCSLNENITVPDTKVNFYAWKRMEPWEPLQLHVDKAVSGLRSLTTLLRALGAQKEAI SPPDAASAAPLRTITADTFRKLFRVYSNFLRGKLKLYTGEACRTGDR
“negative functional modulators of the variant of Erythropoietin (EPO) EV-1-4” means a molecule that binds the nucleotide sequence SEQ ID NO: 4 (called h1-4) ATGGGGGTGCACGAATGTCCTGCCTGGCTGTGGCTTCTCCTGTCCCTGCTGTCG CTCCCTCTGGGCCTCCCAGTCCTGGGCGCCCCACCACGCCTCATCTGTGACAGC CGAGTCCTGGAGAGGTACCTCTTGGAGGCCAAGGAGGCCGAGAATATCACGAC GGGCTGTGCTGAACACTGCAGCTTGAATGAGAATATCACTGTCCCAGACACCAAA GTTAATTTCTATGCCTGGAAGAGGATGGAGGTCGGGCAGCAGGCCCTGTTGGTC AACTCTTCCCAGCCGTGGGAGCCCCTGCAGCTGCATGTGGATAAAGCCGTCAGT GGCCTTCGCAGCCTCACCACTCTGCTTCGGGCTCTGGGAGCCCAGAAGGAAGC CATCTCCCCTCCAGATGCGGCCTCAGCTGCTCCACTCCGAACAATCACTGCTGA CACTTTCCGCAAACTCTTCCGAGTCTACTCCAATTTCCTCCGGGGAAAGCTGAAG CTGTACACAGGGGAGGCCTGCAGGACAGGGGACAGATGA, and the corresponding amino acid sequence SEQ ID NO: 11 MGVHECPAWLWLLLSLLSLPLGLPVLGAPPRLICDSRVLERYLLEAKEAENITTGCAE HCSLNENITVPDTKVNFYAWKRMEVGQQALLVNSSQPWEPLQLHVDKAVSGLRSLT TLLRALGAQKEAISPPDAASAAPLRTITADTFRKLFRVYSNFLRGKLKLYTGEACRTGD R
“negative functional modulators of the variant of Erythropoietin (EPO) EV-1-5” means a molecule that binds the nucleotide sequence SEQ ID NO: 5 (called h1-5) ATGGGGGTGCACGAATGTCCTGCCTGGCTGTGGCTTCTCCTGTCCCTGCTGTCG CTCCCTCTGGGCCTCCCAGTCCTGGGCGCCCCACCACGCCTCATCTGTGACAGC CGAGTCCTGGAGAGGTACCTCTTGGAGGCCAAGGAGGCCGAGAATATCACGAC GGGCTGTGCTGAACACTGCAGCTTGAATGAGAATATCACTGTCCCAGACACCAAA GTTAATTTCTATGCCCTGTTGGTCAACTCTTCCCAGCCGTGGGAGCCCCTGCAGC TGCATGTGGATAAAGCCGTCAGTGGCCTTCGCAGCCTCACCACTCTGCTTCGGG CTCTGGGAGCCCAGAAGGAAGCCATCTCCCCTCCAGATGCGGCCTCAGCTGCTC CACTCCGAACAATCACTGCTGACACTTTCCGCAAACTCTTCCGAGTCTACTCCAA TTTCCTCCGGGGAAAGCTGAAGCTGTACACAGGGGAGGCCTGCAGGACAGGGG ACAGATGA, with the corresponding amino acid sequence SEQ ID NO:12 MGVHECPAWLWLLLSLLSLPLGLPVLGAPPRLICDSRVLERYLLEAKEAENITTGCAE HCSLNENITVPDTKVNFYALLVNSSQPWEPLQLHVDKAVSGLRSLTTLLRALGAQKEA ISPPDAASAAPLRTITADTFRKLFRVYSNFLRGKLKLYTGEACRTGDR
“negative functional modulators of the variant of Erythropoietin (EPO) EV-1-1” means a molecule that binds the nucleotide sequence SEQ ID NO: 6 (called h1-1) ATGGGGGTGCACGAATGTCCTGCCTGGCTGTGGCTTCTCCTGTCCCTGCTGTCG CTCCCTCTGGGCCTCCCAGTCCTGGGCGCCCCACCACGCCTCATCTGTGACAGC CGAGTCCTGGAGAGGTACCTCTTGGAGGCCAAGGAGGCCGAGAATATCACGAC GGGCTGTGCTGAACACTGCAGCTTGAATGAGAATATCACTGTCCCAGGCCCTGTT GGTCAACTCTTCCCAGCCGTGGGAGCCCCTGCAGCTGCATGTGGATAAAGCCGT CAGTGGCCTTCGCAGCCTCACCACTCTGCTTCGGGCTCTGGGAGCCCAGAAGGA AGCCATCTCCCCTCCAGATGCGGCCTCAGCTGCTCCACTCCGAACAATCACTGC TGACACTTTCCGCAAACTCTTCCGAGTCTACTCCAATTTCCTCCGGGGAAAGCTG AAGCTGTACACAGGGGAGGCCTGCAGGACAGGGGACAGATGA
   having the amino acid sequence of SEQ ID NO: 13 MGVHECPAWLWLLLSLLSLPLGLPVLGAPPRLICDSRVLERYLLEAKEAENITTGCAE HCSLNENITVPPGVGQLFPAVGAPAAACG
“negative functional modulators of the variant of Erythropoietin (EPO) EV-2-1” means a molecule that binds the nucleotide sequence SEQ ID NO: 7 (called h2-1) ATGGGGGTGCACGAATGTCCTGCCTGGCTGTGGCTTCTCCTGTCCCTGCTGTCG CTCCCTCTGGGCCTCCCAGTCCTGGGCGCCCCACCACGCCTCATCTGTGACAGC CGAGTCCTGGAGAGGTACCTCTTGGAGGCCAAGGAGGCCGAGAATATCACGAC GGGCTGTGCTGAACACTGCAGCTTGAATGAGAACAATCACTGCTGACACTTTCCG CAAACTCTTCCGAGTCTACTCCAATTTCCTCCGGGGAAAGCTGAAGCTGTACACA GGGGAGGCCTGCAGGACAGGGGACAGATGA, having the amino acid sequence of SEQ ID NO:14 MGVHECPAWLWLLLSLLSLPLGLPVLGAPPRLICDSRVLERYLLEAKEAENITTGCAE HCSLNENNHC.

In one embodiment, said functional negative modulator is an anti-EV3 antibody, preferably a monoclonal antibody developed against an epitope within the amino acid sequence 28-164 of the EV3 variant of human-derived EPO (SEQ ID NO:17). In a further embodiment, said functional negative modulator is a specific monoclonal immunoglobulin purified from hybridoma cells, or is a peptide purified from said mixture by proteolytic cutting of one or more of the immunoglobulins of the mixture, or is another monoclonal antibody generated against an epitope contained in the amino acid sequence of one of the natural or synthetic EPO variants.

In a further preferred embodiment, said functional negative modulator is a monoclonal antibody of one of the EpoR variants, or is a purified specific monoclonal immunoglobulin contained in the antibody mixture obtained from the hybridomas. Alternatively, said negative functional modulator is given by the combination of an anti-V-EPO antibody and anti-V-EpoR antibody.

The therapeutic action of the modulators of the invention can also be exerted through the negative modulation-independent action of the Epo variants by acting on different cellular targets.

The present invention refers to the use of the human erythropoietin (EPO) splicing variants described above.

According to a second aspect, the present invention concerns a negative functional modulator of at least one V-EPO, wherein said variant is selected from the group consisting of: EV3 variant having the amino acid sequence SEQ ID NO: 9, EV4 variant having the amino acid sequence SEQ ID NO: 10, EV1-4 variant having the amino acid sequence SEQ ID NO: 11, EV1-5 variant having the amino acid sequence SEQ ID NO: 12, EV1-1 variant having the amino acid sequence SEQ ID NO: 13, EV2-1 variant having the amino acid sequence SEQ ID NO: 14, for use as a medicament.

According to a third aspect, the present invention concerns a negative functional modulator of at least one variant of EPO, wherein said variant is selected from the group consisting of: EV3 variant with the amino acid sequence SEQ ID NO: 9, EV4 variant having the amino acid sequence SEQ ID NO: 10, EV1-4 variant having the amino acid sequence SEQ ID NO: 11, EV1-5 variant having the amino acid sequence SEQ ID NO: 12, EV1-1 variant having the amino acid sequence SEQ ID NO: 13, EV2-1 variant having the amino acid sequence SEQ ID NO: 14, for use in the treatment of a an oncological pathology, a proliferative pathology, in the therapy of chronic inflammatory diseases on an autoimmune and non-autoimmune basis, of neurodegenerative diseases, and in the treatment of patients undergoing organ or tissue transplantation.

As will be shown in the Examples, tissues of different grade brain tumours, primary glioblastoma stem cells and cell lines of colorectal, prostate, breast, ovarian, lung cancers, neuroblastoma and chronic myeloid leukaemia show an increased expression of the splicing variant of Epo, hs3 and hs4, compared to healthy brain tissues and cells. The negative functional modulators of a natural or synthetic Epo variant, described in the present invention, surprisingly demonstrated to be able to inhibit the proliferation of both stem and endothelial tumour cells isolated from brain tumour biopsies, as well as tumour cell lines from different neoplasms such as colorectal cancer (DLD1), prostate cancer (PC-3, LNCAP), breast cancer (MCF7), chronic myeloid leukaemia (K562), ovarian cancer (A2780) and lung cancer (A549), neuroblastoma (SHSY-5Y) and glioma (T98G). Surprisingly, the negative functional modulators of the V-EPOs showed an important effect on the inhibition of migration in tumour cells and on the formation of vascular structures of primary cancer endothelial cells isolated from glioblastoma biopsies. The treatment with negative functional modulators of the V-EPOs also demonstrated an important effect on the induction of apoptosis in tumour cells. Furthermore, functional negative modulators of Epo receptors (EPOR, EPHB4 and CSF2RB), described in the present invention, surprisingly demonstrated to be able to inhibit the proliferation of cancer stem cells isolated from brain tumour biopsies of tumour cell lines derived from different neoplasms such as colorectal cancer (DLD1), prostate cancer (PC-3, LNCAP), breast cancer (MCF7), chronic myeloid leukaemia (K562), ovarian cancer (A2780) and lung cancer (A549) alone and mostly co-administered with anti-EV3 and FTY720.

It is known that tumour cells, in particular the stem subpopulation, are resistant to pro-apoptotic stimuli. In particular, the biology and aggressiveness of brain tumours makes it possible to model this pathology as an example of a neoplasm in which stem cells play a hierarchical role in modulating the growth of non-stem tumour cells (which constitute the neoplastic mass), for example through the release of S1P with paracrine/autocrine action (see Marfia G, et al. Autocrine/paracrine sphingosine-1-phosphate fuels proliferative and stemness qualities of glioblastoma stem cells. Glia. 2014 December; 62(12):1968-81) and which are by their nature resistant to common chemo-radio therapy treatments, increasing the aggressiveness of the tumour and themselves triggering the relapse. For the purpose of the present invention, primary cell lines from glioblastoma and commercial cell lines from solid tumours and leukaemias that are particularly resistant to current therapeutic standards have been selected.

The functional modulators of the EPO variants aim to sequester and reduce the levels of EV-3, which has a pro-tumour, but not erythropoietic, function, exerted not through binding to the EpoR receptor, since EV-3 lacks the EpoR binding site, but through other pathways, such as the regulation of tumour cell stemness, the resistance to apoptosis, hypoxia and the modulation of S1P synthesis and release.

Preferably said oncological pathology is selected from group consisting of: cerebral astrocytoma, cerebellar astrocytoma, pineal gland astrocytoma, oligodendroglioma, pituitary adenoma, craniopharyngioma, sarcoma, glioblastoma, medulloblastoma, diffuse intrinsic pontine glioma, pituitary adenoma, ependymoma, medulloblastoma, neuroectoderm cancer, neuroblastoma, hypothalamic glioma, mammary cancer, pulmonary cancer, colon cancer, cervical cancer, endometrial cancer, uterine cancer, breast cancer, ovarian cancer, oesophageal cancer, basal cell carcinoma, cholangiocarcinoma, spleen cancer, pancreatic cancer, osteosarcoma, intraocular melanoma, retinoblastoma, stomach cancer, cardiac cancer, liver cancer, hypopharyngeal cancer, laryngeal cancer, cancer of the oral cavity, nasal and paranasal cancer, salivary gland cancer nasopharyngeal cancer, throat cancer, thyroid cancer, pancreatic cancer, renal cancer, prostate cancer, rectal cancer, testicular cancer, melanoma, mesothelioma, pheochromocytoma, haematological cancers, lung cancer and acute or chronic myeloid leukaemia.

According to a fourth aspect, a V-EPO is described which is selected from the group consisting of: EV3 variant with the amino acid sequence SEQ ID NO: 9, EV4 variant having the amino acid sequence SEQ ID NO: 10, EV1-4 variant having the amino acid sequence SEQ ID NO: 11, EV1-5 variant having the amino acid sequence SEQ ID NO: 12, EV1-1 variant having the amino acid sequence SEQ ID NO: 13, EV2-1 variant having the amino acid sequence SEQ ID NO: 14, for use as a diagnostic agent.

Preferably said diagnostic agent is for the molecular diagnosis in oncology, for prognostic purposes and for the personalisation of the therapy.

Surprisingly, it was seen that said negative functional modulator of at least one erythropoietin variant can be used as a diagnostic agent for the measurement of the tissue or circulating levels of an EPO variant and can be advantageously associated with the measurement of the expression levels of the receptors EPO, EPOR, EPHB4, CSF2RB or the promoter methylation of the same genes.

Preferably, the diagnostic use is in the following pathologies: oncological pathology selected from group consisting of: brain astrocytoma, cerebellar astrocytoma, pineal gland astrocytoma, oligodendroglioma, pituitary adenoma, craniopharyngioma, sarcoma, glioblastoma, medulloblastoma, diffuse intrinsic pontine glioma, pituitary adenoma, ependymoma, medulloblastoma, neuroectodermal tumour, neuroblastoma, hypothalamic glioma, mammary tumour, pulmonary tumour, colon cancer, cervical cancer, endometrial cancer, uterine cancer, breast cancer, ovarian cancer, oesophageal cancer, basal cell cancer, cholangiocarcinoma, spleen cancer, pancreatic cancer, osteosarcoma, intraocular melanoma, retinoblastoma, stomach cancer, cardiac cancer, liver cancer, hypopharyngeal cancer, laryngeal cancer, cancer of the oral cavity, nasal and paranasal cancer, salivary gland cancer, nasopharyngeal cancer, throat cancer, thyroid cancer, pancreatic cancer, renal cancer, prostate cancer, rectal cancer, testicular cancer, melanoma, mesothelioma, pheochromocytoma, haematological cancers, lung cancer and acute or chronic myeloid leukaemia, proliferative pathologies, chronic inflammatory diseases on an autoimmune and non-autoimmune basis, of neurodegenerative diseases, and in the treatment of patients undergoing organ or tissue transplantation.

In a preferred embodiment, said oncological pathology is glioblastoma, grade II fibrillary, protoplasmic, gemistocytic astrocytoma and grade III anaplastic astrocytoma, including gliomatosis cerebri.

More preferably, said oncological pathology is drug-resistant.

According to a fifth aspect, a pharmaceutical composition is described, comprising a negative functional modulator of at least one variant of the erythropoietin (EPO) selected from the group consisting of: EV3 variant having the amino acid sequence SEQ ID NO: 9, EV4 variant having the amino acid sequence SEQ ID NO: 10, EV1-4 variant having the amino acid sequence SEQ ID NO: 11, EV1-5 variant having the amino acid sequence SEQ ID NO: 12, EV1-1 variant having the amino acid sequence SEQ ID NO: 13, EV2-1 variant having the amino acid sequence SEQ ID NO: 14, and/or anti Epo receptor (EpoR, EPHB4, CSF2RB), an antisense oligonucleotide, DNA decoy, RNA decoy, a ribozyme, an antagomiR, a shRNA, LNA or siRNA, for use in the treatment of an oncological pathology, a proliferative pathology, chronic inflammatory diseases on an autoimmune and non-autoimmune basis, and in the treatment of patients undergoing organ or tissue transplantation.

Since the molecule of the present invention exhibits low toxicity, it can be safely administered alone or as a pharmaceutical formulation as tablets, powder, granules, capsules (including soft capsules), liquid agents, injections, suppositories or slow-release agents generally used for the production of pharmaceutical preparation, orally or parenterally (topically, rectally, intravenously, subcutaneously, intramuscularly, intranasally, intravaginally, through the oral mucosa, pulmonary mucosa or by transocular administration, etc.) or by incorporation into liposomes or by functional targeting specific targets or compartments, e.g. intracranial, intratumoral, by binding to cellular, molecular and chemical vectors or in association with molecules that allow the temporary opening of the Blood-Brain Barrier, (e.g. mannitol) or other anti-inflammatory drugs, monoclonal antibodies and immunosuppressive drugs.

Where the therapeutic target are brain tumour cells, said combination is formulated in such a way as to facilitate the overcoming thereof of the blood brain barrier. By way of example, factors known as carriers, such as IL13/IL13R-ApoE, are used in said formulation. Alternatively, said compounds are administered in the form of nanoparticles or liposomes, or by local or intrathecal treatment, or wafers of biodegradable biopolymers loaded with the therapy of interest are implanted directly into the surgical cavity after resection surgery of both primary tumours and relapses.

Alternatively, it can be administered by using all those technologies currently present linked to gene therapy, such as the use of vectors for the introduction of nucleic acids into the patient's cells. This administration can be done either systemically, i.e. by infusion, or locally, by administering vectors directly to the site of the tumour lesion. Preferably, said composition further comprises a therapeutically effective amount of one or more natural or synthetic molecules acting on S1P receptors, and/or on S1P metabolism directly or indirectly, and/or antiblastic-cytotoxic and/or antiviral and/or antiangiogenic molecules. Even more preferably, said molecule acting on S1P receptors, and/or on S1P metabolism directly or indirectly, is FTY720 or analogues thereof. Preferably, said antiblastic-cytotoxic and/or antiviral and/or antiangiogenic molecule is selected from the group comprising: paclitaxel, taxol, cycloheximide, carboplatin, chlorambucil, cisplatin, colchicine, cyclophosphamide, daunorubicin, dactinomycin, diethylstilbestrol, doxorubicin, etoposide, 5-fluorouracil, floxuridine, melphalan, methotrexate, mitomycin, 6-mercaptopurine, teniposide, 6-thioguanine, vincristine and/or vinblastine, photoemustine, carmustine, irinotecan systemically or carmustine-adsorbed biopolymer wafers for locoregional therapy, temozolomide, tamoxifen, valganciclovir, ganciclovir, acyclovir, anti-VEGF, anti-VEGFR, Anti-HER2/neu, Anti-EGFR, gefitinib, bevacizumab, ranibizumab, Vatalanib, Cediranib, Sorafenib, Sunitinib, Motesanib, Axitinib, metformin, drugs for immunotherapy.

According to a sixth aspect, a pharmaceutical kit is described comprising two or more components selected from the group, consisting of:
A negative functional modulator of at least one variant of the erythropoietin (EPO), wherein said variant is selected from the group consisting of: EV3 variant having the amino acid sequence SEQ ID NO: 9, EV4 variant having the amino acid sequence SEQ ID NO: 10, EV1-4 variant having the amino acid sequence SEQ ID NO: 11, EV1-5 variant having the amino acid sequence SEQ ID NO: 12, EV1-1 variant having the amino acid sequence SEQ ID NO: 13, EV2-1 variant having the amino acid sequence SEQ ID NO: 14;
a monoclonal antibody to at least one of the at least one variant of the erythropoietin (EPO), wherein said variant is selected from the group consisting of: EV3 variant having the amino acid sequence SEQ ID NO: 9, EV4 variant having the amino acid sequence SEQ ID NO: 10, EV1-4 variant having the amino acid sequence SEQ ID NO: 11, EV1-5 variant having the amino acid sequence SEQ ID NO: 12, EV1-1 variant having the amino acid sequence SEQ ID NO: 13, EV2-1 variant having the amino acid sequence SEQ ID NO: 14, wherein said antibody is a bispecific antibody, diabody, tryabody, or tetrabody;
a polynucleotide encoding a variant of the erythropoietin (EPO), wherein said variant is selected from the group consisting of: EV3 variant having the amino acid sequence SEQ ID NO: 9, EV4 variant having the amino acid sequence SEQ ID NO: 10, EV1-4 variant having the amino acid sequence SEQ ID NO: 11, EV1-5 variant having the amino acid sequence SEQ ID NO: 12, EV1-1 variant having the amino acid sequence SEQ ID NO: 13, EV2-1 variant having the amino acid sequence SEQ ID NO: 14;
a chemotherapy drug;
an antiviral drug;
a negative functional modulator of a natural or synthetic variant of the Epo receptors (EpoR, EPHB4, CSF2RB);
a negative functional modulator of Epo receptors (EpoR EPHB4, CSF2RB);
a polynucleotide encoding Epo receptors (EpoR EPHB4, CSF2RB);
a negative functional modulator of the sphingosine-1-phosphate (S1P) signalling pathway;
an inhibitor of S1P and the metabolism thereof; or
EPO mimetics that preserve the erythropoietic function.

In the present invention, “negative functional sphingosine-1-phosphate modulator” means a therapeutically effective amount of one or more natural or synthetic molecules acting on S1P receptors, and/or on S1P metabolism directly or indirectly.

Preferably, said molecule acting on S1P receptors, and/or on S1P metabolism directly or indirectly, is FTY720 or analogues thereof.

Preferably, said chemotherapeutic drug is paclitaxel, taxol, cycloheximide, carboplatin, chlorambucil, cisplatin, colchicine, cyclophosphamide, daunorubicin, dactinomycin, diethylstilbestrol, doxorubicin, etoposide, 5-fluorouracil, floxuridine, melphalan, methotrexate, mitomycin, 6-mercaptopurine, teniposide, 6-thioguanine, vincristine and/or vinblastine, photoemustine, carmustine systemically or carmustine-adsorbed biopolymer wafers for loco-regional therapy, temozolomide, tamoxifen, or is a biological drug anti-VEGFR, Anti-HER2/neu, Anti-EGFR or gefitinib, regorafenib, nivolumab, metfomine, immunotherapy drug. Preferably said antiviral drug is valganciclovir, ganciclovir or acyclovir.

According to a seventh aspect, a monoclonal antibody to at least one of the variants of the erythropoietin (EPO) is described, wherein said variant is selected from the group consisting of: EV3 variant having the amino acid sequence SEQ ID NO: 9, EV4 variant having the amino acid sequence SEQ ID NO: 10, EV1-4 variant having the amino acid sequence SEQ ID NO: 11, EV1-5 variant having the amino acid sequence SEQ ID NO: 12, EV1-1 variant having the amino acid sequence SEQ ID NO: 13, EV2-1 variant having the amino acid sequence SEQ ID NO: 14.

Preferably said antibody is a bi-specific antibody, a diabody, tryabody, or tetrabody antibody.

According to an eighth aspect, a negative functional modulator of at least one variant of the erythropoietin (EPO) is described, wherein said variant is selected from the group consisting of: EV3 variant having the amino acid sequence SEQ ID NO: 9, EV4 variant having the amino acid sequence SEQ ID NO: 10, EV1-4 variant having the amino acid sequence SEQ ID NO: 11, EV1-5 variant having the amino acid sequence SEQ ID NO: 12, EV1-1 variant having the amino acid sequence SEQ ID NO: 13, EV2-1 variant having the amino acid sequence SEQ ID NO: 14, for use as a diagnostic agent.

Preferably, said diagnostic agent is for the molecular diagnosis in oncology, for prognostic purposes and for the personalisation of the therapy.

Surprisingly, it was seen that said negative functional modulator of at least one erythropoietin variant can be used as a diagnostic agent for the measurement of the tissue or circulating levels of an EPO variant and can be advantageously associated with the measurement of the expression levels of the receptors EPO, EPOR, EPHB4, CSF2RB or the promoter methylation of the same genes.

In the Examples it will be shown that in brain tumours there is a genetic profile in which the EPO and EPHB4 genes, positioned at the Chr7q cytoband level, are present in an amplification state in 68% of the tumour biopsies analysed (Example 1). In addition, it will be described that tumour cells isolated from tumours of different grade and origin show an increased expression of the Epo splicing variant, hS3, and to a lesser extent hs4, compared to healthy brain cells and to mesenchymal cells isolated from adipose tissue (Example 2). It will also be shown that Epo receptors have a different expression in tumour cells compared to healthy cells and human adipose tissue cells.

In addition, as can be seen from Example 3, negative functional modulators of Epo variants surprisingly demonstrated to be able to inhibit the proliferation of tumour cells, both stem cells and those isolated from biopsies of brain tumours, as well as tumour cell lines deriving from different neoplasms.

It was surprisingly seen that the negative functional modulators of V-EPOs demonstrated an important action on the inhibition of the migration in GEC cells (Example 4) and in the formation of vascular structures (Example 5). To date, the latter is still considered a functional test for the evaluation of anti-tumour and/or antiangiogenic treatments. The compounds are able to act simultaneously on the EPO signalling pathway without affecting the erythropoietic activity thereof.

Finally, in Example 6, negative functional modulators of EPOR, EPHB4 and CSF2RB were able to inhibit the growth of cancer stem cells isolated from brain biopsies and tumour lines with different efficacy alone or in co-administration with anti-EV3 and/or FTY720.

The results obtained with the negative functional modulators of V-Epo, precisely because they were obtained in an in vitro model characterised by a strong resistance to apoptotic stimuli, demonstrate the surprising effectiveness of these modulators.

The combined treatment carried out on cancer stem cells with anti-EV3 antibody and FTY720 and/or temozolomide (in the case of cells from brain tumour biopsies) showed a superior effect, in terms of inducing apoptosis and blocking tumour growth compared to the effect measured with anti-EV3, FTY720 and temozolomide tested individually.

FTY720, a functional agonist for the S1P receptor, is activated following phosphorylation mediated by sphingosine kinases, SK1 and SK2 and is used in post-transplant immunosuppressive therapy, in multiple sclerosis and also in the treatment of neoplasms. The results obtained with the co-administration of anti-EV3 and FTY720 showed superior results, favouring the combination of anti-EV3 and FTY720, the latter being administered in the form of pro-drug and activated at the cellular level by Sk1 and SK2. Or the combination with anti-EV3 and one or more molecules selected from S1P antagonists comprising FTY720-P, anti-S1P, SW-2871, VPC24191, AUY954, SEW2871 (5-[4-phenyl-5-(trifluoromethyl)-2-thienyl]-3-[trifluoromethyl)phenyl]-1,2,4-oxadiazole), VPC23153, DS-GS-44, VPC01091. In addition, the results obtained by administering negative functional modulators of EpoR, EPHB4 and CSF2RB proved to be effective in inhibiting the growth of brain tumour stem cells and tumour cell lines of different origins. The co-administered treatment with FTY720 showed superior results. The following examples of embodiments of the present invention are given below by way of illustration.

### EXAMPLES

### Example 1: Tumour Tissue Genetics, Biological Models and Treatments

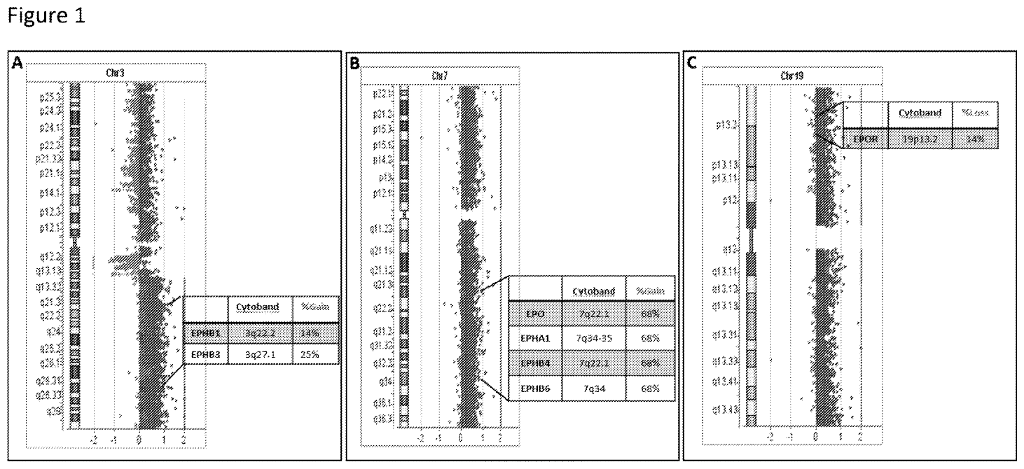

The results of the analyses of chromosomal alterations such as Copy Number Variation (CNV) showed that in neoplasm brain tissues there is a chromosomal imbalance in favour of the EPO signalling pathway genes. In particular, in tumour biopsies from patients affected by GBM, the EPHB1 and EPHB3 genes were amplified in 14% and 25% of cases respectively (FIG. 1A); the EPO, EPHA1, EPHB4 and EPHB6 genes (FIG. 1B) were amplified in 68% of the tumour biopsies. In contrast, the EPOR gene, if altered, always resulted in deletion in 14% of the brain tumour biopsies analysed (FIG. 1C). The genomic DNA was extracted from tissue samples using the QIAamp Fast DNA Tissue kit according to the indicated protocol (Qiagen). DNA was quantified by means of a NanoDrop ND-1000 spectrophotometer (ThermoFisher Scientific) and the integrity was assessed by microcapillary electrophoresis on Bioanalyzer 2100 (Agilent Technologies). The High-resolution analysis of CNVs was performed using an 8×60K array platform (Agilent Technologies). The results were interpreted by means of the software Feature Extraction and Agilent CytoGenomics v. 4.0.3.12 and Genomic Workbench v. 7.0.4.0 (Agilent Technologies). Raw data were analysed using Cytogenomics software with the ADM-2 algorithm (breakpoint positions were reported according to the GRch37/hg19 reference genome). A minimum of three consecutive probes/regions was considered as a filter.

Table 1 summarises the results of the percentage of chromosomal alterations in EPO signalling pathway genes, as shown in FIGS. 1A, 1B and 1C.

| | | % GBM | % GBM |
|---|---|---|---|
| Gene | Cytoband | Gain | Loss |
| EPO | 7q22.1 | 68 | — |
| EPOR | 19p13.2 | — | 14 |
| EPHA1 | 7q34-q35 | 68 | — |
| EPHB1 | 3q22.2 | 14 | — |
| EPHB | 3q27.1 | 25 | — |
| EPHB4 | 7q22.1 | 68 | — |
| EPHB6 | 7q34 | 68 | — |
| CD131 | 22q12.3 | — | 21 |

The following biological models were used:
a) Primary low-grade glioma cancer stem cells (LG-CSCs) isolated from tumour biopsies;
b) Primary glioblastoma cancer stem cells (GCSc) isolated from tumour biopsies;
c) Primary glioblastoma endothelial tumour cells (GECs) isolated from tumour biopsies;
d) Primary glioblastoma tumour cells isolated from the whole tumour mass, differentiated tumour control cells (GBM-CT);
e) DLD-1 cell line, colorectal adenocarcinoma cells. Epo has been shown to have a serious negative effect in promoting the neoplastic process in colon cancer by enhancing carcinogenesis through increased expression of EpoR;
f) PC-3 and LNCAP cell line, human prostate tumour cells used in cancer research and drug development. Human hepatocellular (Eph) erythropoietin-producing receptors have been reported to be overexpressed and associated with poor prognosis and reduced survival in prostate cancer patients, and are considered as predictive markers of the aggressive prostate cancer behaviour. Furthermore, it has recently been shown that in LNCAP, the simultaneous overexpression of Epo and EpoR in resistant prostate cancer plays an important role in progression and is responsible for the development of a neuroendocrine phenotype;
g) MCF-7 cell line, breast tumour cells. This cell line has been shown to be reactive to the treatment with human erythropoietin (rHuEPO) in terms of increased cell proliferation;
h) K562 cell line, chronic myeloid leukaemia cells. Circulating levels of Epo are reliably higher in myelodysplastic syndromes than in healthy people, with a negative predictive role;
i) A2780 cell line, ovarian tumour cells used in toxicity testing, drug screening and cancer genetic studies. Previous studies have shown that A2780s express EPOR and that the treatment with Epo resulted in increased resistance to chemotherapy.
l) A549 cell line, human lung tumour cells used for screening drug efficacy, biochemical mechanisms and alveolar differentiation;
m) SHSY-5Y line is a cell line isolated from a bone marrow biopsy taken from a patient with neuroblastoma. SHSY cells are often used as in vitro models of oncological pathology and neuronal differentiation.
n) ASTROCITI line is a commercial cell line of human astrocytes

The following treatments were tested on the cell models listed above to assess the anti-tumour efficacy in terms of cell viability, induction of apoptosis, cell migration and angiogenesis:
1. Anti-EV3 treatment, obtained by hybridoma technique, which specifically recognises the amino acid sequence of the hs3 variant of Epo;
2. Treatment with Temozolomide (TMZ), used as a standard treatment in patients with glioblastoma;
3. Treatment with Fingolimod (FTY720), used as a functional S1P antagonist;
4. Combinations of the treatments described above.
5. treatment with anti-EPOR
6. treatment with anti-EPHB4
7. treatment with anti-CSF2RB
8. Combination of the treatment with anti-EPOR, anti-EV3 and/or FTY720
9. Combination of the treatment with anti-EPHB4, anti-EV3 and/or FTY720
10. Combination of the treatment with anti-CSF2RB, anti-EV3 and/or FTY720

### Example 2: Quantitative PCR Analysis in Human Tumour Tissues and Cells

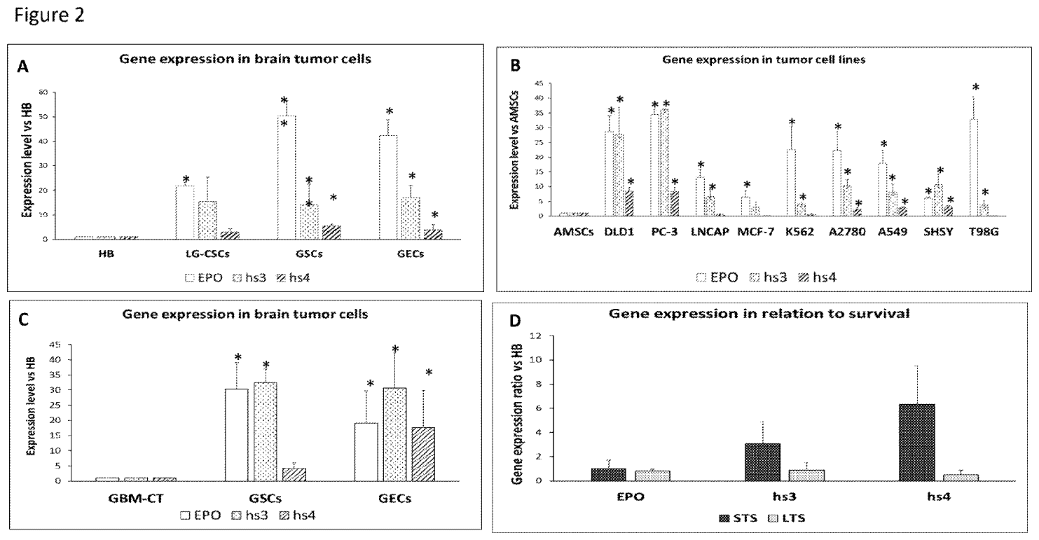

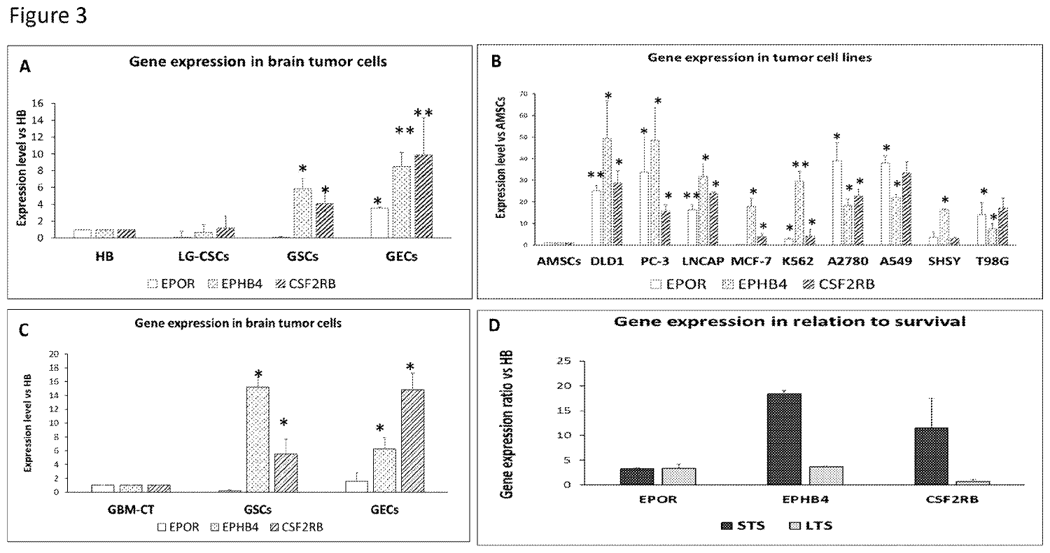

The gene expression of EPO, of the variants thereof, hs3, hs4, and of the receptors EPOR, EPHB4, CSF2RB was assessed by means of Real-Time PCR technique (FIGS. 2 and 3).

Total RNA from paraffin-embedded human tissue (FFPE) was isolated using the High Pure RNA Paraffin Kit (Sigma-Aldrich) and RNA from cells was isolated using the RNeasy Plus Kit (Qiagen).

Total RNA from human brain tissue was purchased from TakaraBio (Japan). RNA reverse transcription was performed on RNA (1 μg) with Superscript II, Rnase Out, random primers and dNTPs. EPOwt transcription-specific primers (SPEC_EPO; FWD: 5′-GTGCTGAACACTGCAGCTTG-3′ (SEQ ID NO: 19); REV: 5′-CAGACTTCTACGGCCTGCTG-3′) (SEQ ID NO: 20), for the hs3 transcription (SPEC_hs3; FWD: 5′-CCGAGAATATCACGGTCGGG-3′ (SEQ ID NO: 21) REV: 5′-CGGCTTTATCCACATGCAGC-3′) (SEQ ID NO: 22), for the hs4 transcription (SPEC_hs4; FWD: 5′-GAAGAGGATGGAGCCGTGG-3′ (SEQ ID NO: 23) REV: 5′-ATCTGGAGGGGAGATGGCTT-3′) (SEQ ID NO: 24) for the EPOR transcription (FWD: 5′-ATCCTGACGCTCTCCCTCAT-3′ (SEQ ID NO: 25) REV: 5′-AGGCCTTCAAACTCGCTCTC-3′), (SEQ ID NO: 26), for the EPHB4 transcription (FWD: 5′-GTCCCGCGCGGAGTATC-3′ (SEQ ID NO: 27) REV: 5′-CTTCTCCTG ACAGGGGCTTG-3′) (SEQ ID NO: 28), for the CSF2RB transcription (FWD: 5′-AGTGGGAGGAGAAGATCCCC-3′ (SEQ ID NO: 29) REV: 5′-CCCAGGATGTCAGGTAGGGA-3′) (SEQ ID NO: 30) were ordered at Metabion international AG GmbH (Germany). Real-Time PCR reactions were performed with the StepOnePlus™ Real-Time PCR System instrument (Applied Biosystems) using Power SYBR™ Green PCR Master Mix (Applied Biosystems). The melting curve analysis was performed to check for the presence of non-specific products.

FIG. 2 shows the results of experiments of EPO gene expression and the hs3 and hs4 variants thereof in cancer stem cells and commercial and non-commercial tumour cells (FIGS. 2A and 2C). Primary glioblastoma stem cells and colorectal, prostate, breast, ovarian, lung cell lines and the chronic myeloid leukemia and neuroblastoma cell line show an increased expression of the Epo splicing variant, hs3, and hs4 compared to healthy brain cells and mesenchymal cells isolated from adipose tissue (FIGS. 2A and 2B, respectively). Surprisingly, the results show that the subpopulation of stem cells and endothelial cells isolated from brain tumour biopsies express statistically significant high levels of EPO, hS3 and hs4 compared to the differentiated tumour cells composing the tumour mass (FIG. 2C). In addition, an analysis was performed to investigate whether the gene expression of EPO, hs3 and hs4, correlated with a different survival. The results showed that short term survival (STS) patients expressed higher levels of hs3 and hs4, compared to long term survival patients (LTS, survival of more than 12 months, FIG. 2D)). These data confirm that there is an enrichment in the natural variants of EPO, hs3 and hs4, in tumour cells compared to healthy cells and more so in cancer stem and endothelial cells than in differentiated cells composing the tumour mass. Surprisingly, the results showed that hs3 and hs4 may play a predictive role in the pathology.

Regarding the gene expression of EPO receptors, the results show that there are high expression levels of EPHB4 and CSF2RB in stem and endothelial cells isolated from brain tumours, compared to healthy cells, and in tumour cells compared to mesenchymal cells isolated from adipose tissue (FIGS. 3A and 3B, respectively). Surprisingly, EPHB4 and CSF2RB receptors were significantly more expressed in cancer stem and endothelial cells than in differentiated cells of the tumour mass, in contrast to the EPOR receptor which was found to be less expressed (FIG. 3C). Furthermore, the results show that EPHB4 and CSF2RB may play a predictive role in the pathology, as STS patients show higher expression levels of EPHB4 and CSF2RB than long survival patients (FIG. 3D).

### Example 3: Cell Viability Analysis

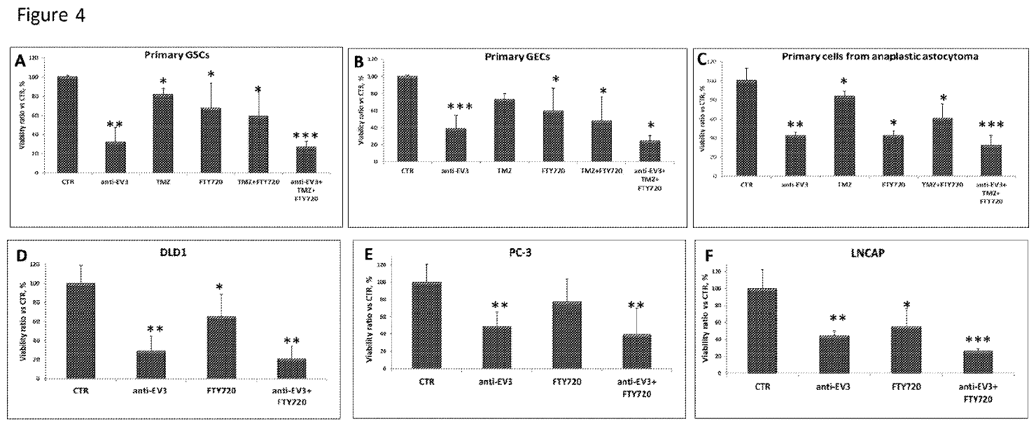

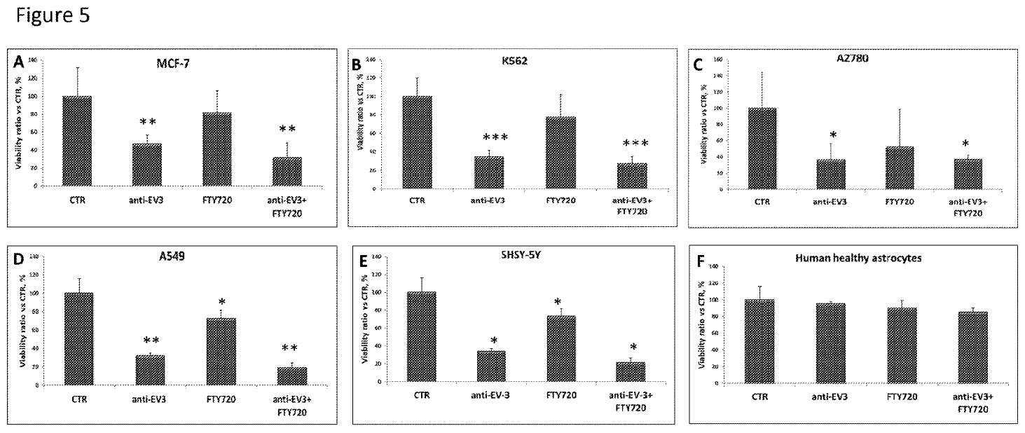

Cell viability was assessed by assaying the dosage of 3-(4,5-dimethylthiazol-2-yl)-2,5-diphenyl-tetrazolium bromide (MTT), as a function of the redox potential (FIGS. 4 and 5). The cells were plated at a density of 5×10³ cells/well in a 96-well plate and cultured for 24 hours in a basal growth medium, now referred to as “Basal Medium” or BM. After 24 hours, the culture media were replaced with fresh BM media as a control condition (CTR) or containing the following treatments:
1) CTR (control condition, replacement of the culture medium at time 0 with fresh culture medium);
2) anti-EV3 (the culture medium was replaced at time 0 with a fresh culture medium containing anti-EV3 antibody at 6 μg/mL);
3) TMZ (the culture medium was replaced at time 0 with a fresh culture medium containing TMZ 100 μM);
4) FTY720 (the culture medium was replaced at time 0 with a fresh culture medium containing FTY720 100 nM);
5) TMZ+FTY720 (the culture medium was replaced at time 0 with a fresh culture medium containing TMZ 100 μM and FTY720 100 nM);
6) anti-EV3+TMZ+FTY720 (the culture medium was replaced at time 0 with a fresh culture medium containing anti-EV3 6 μg/mL, TMZ 100 μM and FTY720 100 nM);
7) anti-EV3+FTY720 (the culture medium was replaced at time 0 with a fresh culture medium containing anti-EV3 6 μg/mL and FTY720 100 nM).

The tests were performed in triplicate after 96 hours of treatment, at the end of which the culture media were replaced with 100 μL of fresh medium and 10 LI of MTT 5 mg/mL in D-PBS. After 4 hours incubation, the media were removed and the cells were lysed with 100 μL of 2-propanol/formic acid (95:5, by volume) for 10 min. The absorbance was then read at 570 nm in the microplate reader.

In particular, after 96 hours of treatment with anti-EV3, the tumour cells showed a mortality of 60%, which increased up to 70% with combined treatment. *P<0.05, **P<0.01 ***P<0.001 compared to CTR for all treatments tested.

It was therefore possible to verify that the negative functional modulators of a natural or synthetic variant of Epo surprisingly demonstrated to be able to inhibit the proliferation of tumour cells, both stem cells (FIG. 4A) and endothelial cells (FIG. 4B) and those derived from an anaplastic astrocytoma brain tissue (FIG. 4C). In addition, the negative functional modulators of a natural or synthetic variant of Epo surprisingly demonstrated to be able to inhibit the proliferation of tumour cell lines from different neoplasms such as colorectal cancer (DLD1, FIG. 4D), prostate cancer (PC-3, FIG. 4E, LNCAP, FIG. 4F), breast cancer (MCF7, FIG. 5A), chronic myeloid leukaemia (K562, FIG. 5B), ovarian cancer (A2780, FIG. 5C), lung cancer (A549, FIG. 5D), neuroblastoma (FIG. 5E). In contrast, the aforesaid treatments had no effect on the viability of human astrocytes treated with the same conditions and timing as the tumour cells (FIG. 5F).

### Example 4: Migration Tests on GECs and DLD1

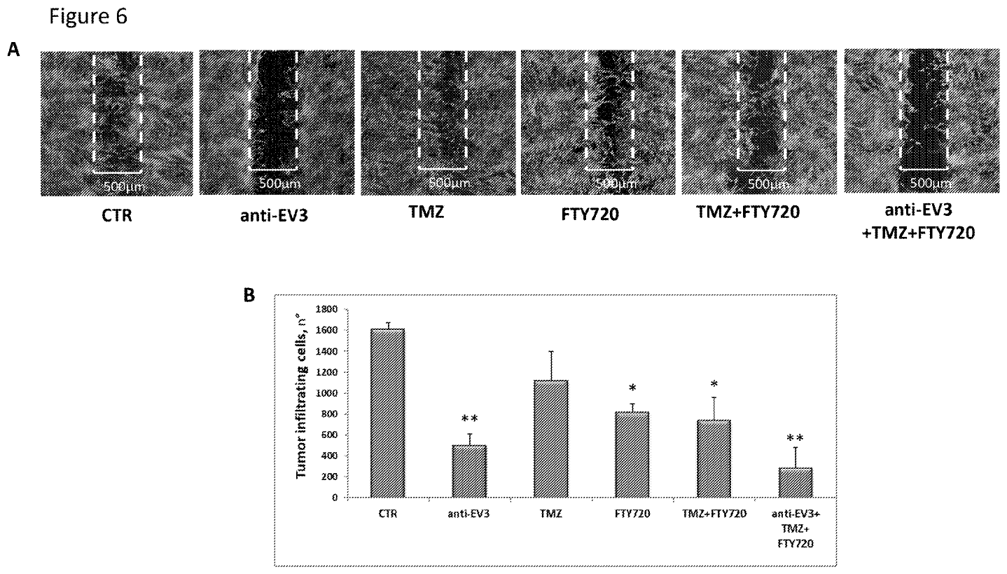

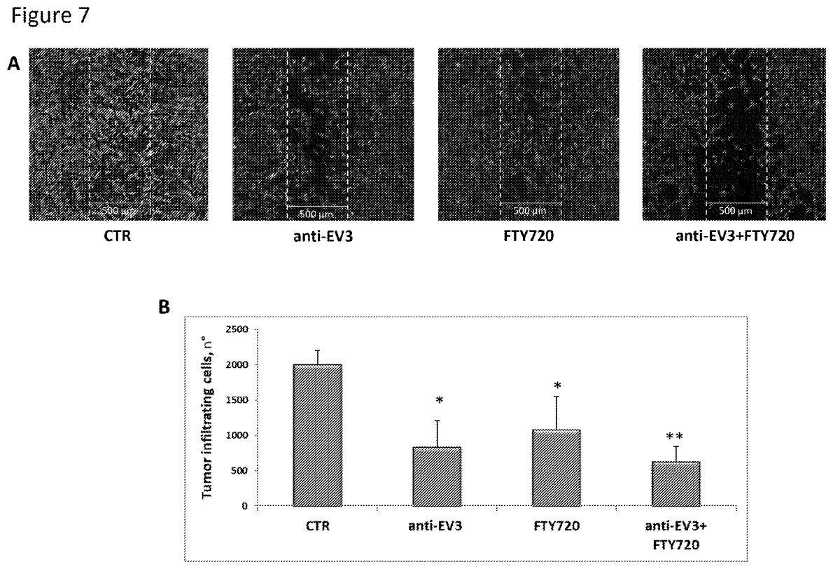

Cells (2×10⁴) were plated in the BM insert compartments and cultured at 37° C., 5% CO₂ and 5% O₂. After 24 hours, the insert was removed and the cells were cultured for further 48 hours under control conditions (CTR) or in the presence of the above treatments. After 48 hours, the cells were stained with CalceinAM at 1 μg/mL (Invitrogen) and the images were acquired with a Leica DMI6000B inverted microscope (Leica Microsystems) equipped for time-lapse videomicroscopy in five random fields (FIG. 6A). The cells migrated in space were counted using ImageJ/Analyse Particles (FIG. 6B). The same tests were performed on DLD1 cells (FIGS. 7A and 7B). Following treatments with anti-EV3, in both GECs and DLD1 there was an average 61% decrease in migration, which increased surprisingly up to 73% after combined treatment. The data are expressed as mean±standard deviation of at least 3 experiments in triplicate. *P<0.05, **P<0.01 ***P<0.001 compared to CTR for all treatments tested.

FIGS. 6 and 7 show that the negative functional modulators of EPO variants demonstrated an important action on the migration inhibition in GEC (FIG. 6) and DLD1 cells (FIG. 7).

### Example 5: Functional Test of the Formation of New Vascular Structures

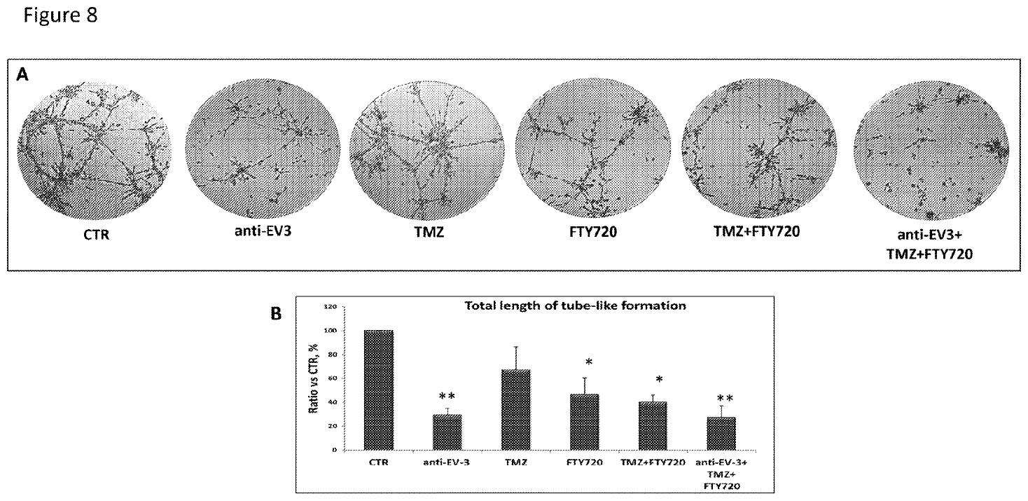

GECs (1×10⁴) were plated on 10 μL of Matrigel in BM as a control condition (CTR) or in media containing the above treatments and cultured at 37° C., 5% CO₂, 5% O₂. After 48 hours, the formation of tubular structures was assessed by phase-contrast microscopy. It was observed that the anti-EV3 treatment is able to block the formation of tubular structures (FIG. 8A). The total tube length, measured using the ImageJ's Angiogenesis Analyzer plug-in, shows a decrease by 63% after administration of anti-EV3 treatments and 80% after administration of the combined anti-EV3+TMZ+FTY720 treatments (FIG. 8B). The data are expressed as mean±standard deviation of at least 3 experiments in triplicate copy. *P<0.05, **P<0.01 ***P<0.001 compared to CTR for all treatments tested.

The negative functional modulators of the EPO variants have been shown to have an important effect on the formation of vascular structures in primary tumour endothelial cells isolated from glioblastoma biopsies (FIG. 8).

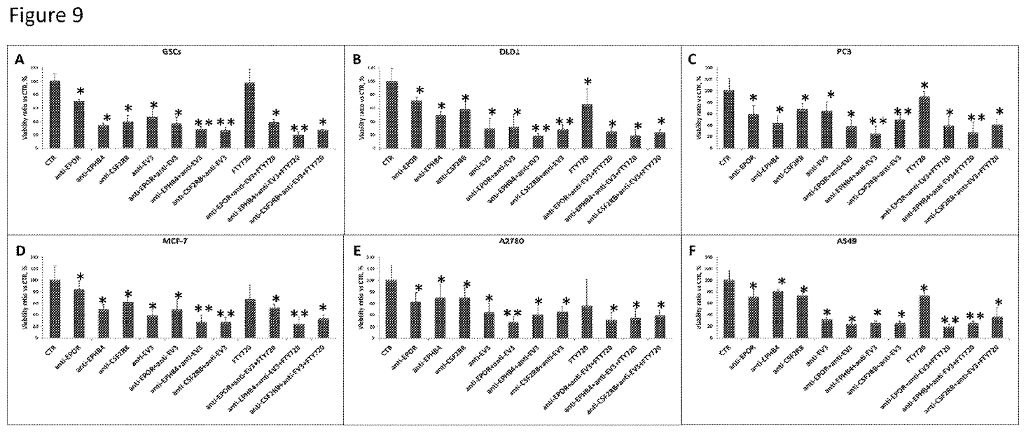

After 96 hours of treatment with anti-EPOR, anti-EPHB4, anti-CSF2RB, the glioblastoma tumour cells showed a mortality of 60%, 40% and 45% respectively, which increased up to 70% with combined treatment with anti-EV3 and/or with FTY720. *P<0.05, **P<0.01 ***P<0.001 compared to CTR for all treatments tested (FIG. 9).

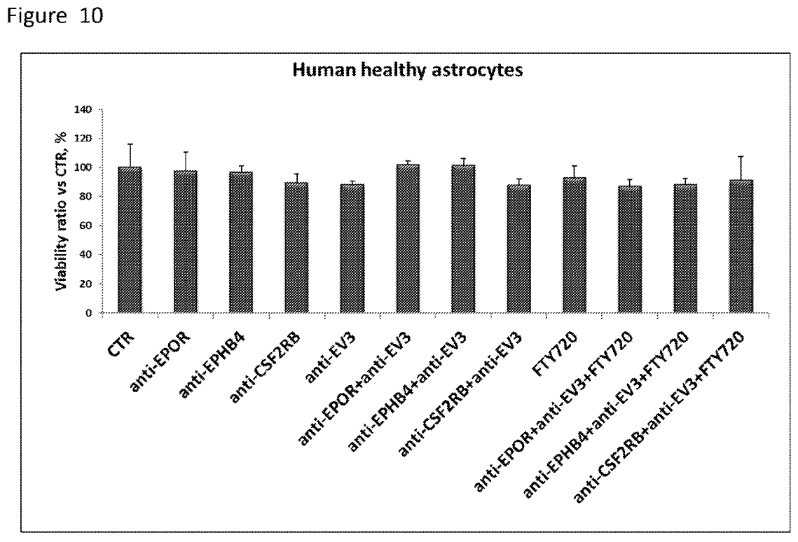

It was therefore possible to verify that the negative functional modulators of Epo receptors surprisingly demonstrated to be able to inhibit the proliferation of tumour cells, both stem cells (FIG. 9A) and tumour cell lines deriving from different neoplasms such as colorectal cancer (DLD1, FIG. 9B), prostate cancer (PC-3, FIG. 9C), breast cancer (MCF7, FIG. 9D), ovarian cancer (A2780, FIG. 9E) and lung cancer (A549, FIG. 9F). Anti-EPOR, anti-EPHB4, anti-CSF2RB treatments alone or co-administered with FTY720 and/or anti-EV3 did not show any significant effect on the commercial line of human astrocytes (FIG. 10).

1. A negative functional modulator of at least one variant of the erythropoietin (EPO), wherein said variant is selected from the group consisting of: EV3 variant having the amino acid sequence SEQ ID NO: 9, EV4 variant having the amino acid sequence SEQ ID NO: 10, EV1-4 variant having the amino acid sequence SEQ ID NO: 11, EV1-5 variant having the amino acid sequence SEQ ID NO: 12, EV1-1 variant having the amino acid sequence SEQ ID NO: 13, EV2-1 variant having the amino acid sequence SEQ ID NO: 14, wherein said negative functional modulator is an antibody anti-EV3, anti-EV4, anti-EV1-4, anti-EV1-5, anti-EV1-1, or EV2-1, or anti-Epo receptor (EpoR) anti-EPHB4, anti-CSF2RB, an antisense oligonucleotide, DNA decoy, RNA decoy, a ribozyme, an antagomiR, a shRNA, LNA or siRNA.

2. A negative functional modulator of at least one variant of the erythropoietin (EPO), wherein said variant is selected from the group consisting of: EV3 variant having the amino acid sequence SEQ ID NO: 9, EV4 variant having the amino acid sequence SEQ ID NO: 10, EV1-4 variant having the amino acid sequence SEQ ID NO: 11, EV1-5 variant having the amino acid sequence SEQ ID NO: 12, EV1-1 variant having the amino acid sequence SEQ ID NO: 13, EV2-1 variant having the amino acid sequence SEQ ID NO: 14, for use as a medicament.

3. A negative functional modulator of at least one variant of the erythropoietin (EPO), wherein said variant is selected from the group consisting of: EV3 variant having the amino acid sequence SEQ ID NO: 9, EV4 variant having the amino acid sequence SEQ ID NO: 10, EV1-4 variant having the amino acid sequence SEQ ID NO: 11, EV1-5 variant having the amino acid sequence SEQ ID NO: 12, EV1-1 variant having the amino acid sequence SEQ ID NO: 13, EV2-1 variant having the amino acid sequence SEQ ID NO: 14, for use in the treatment of a an oncological pathology, a proliferative pathology, in the therapy of chronic inflammatory diseases on an autoimmune and non-autoimmune basis, of neurodegenerative diseases, and in the treatment of patients undergoing organ or tissue transplantation.

4. The modulator for use according to claim 3, wherein said oncological pathology is selected from group consisting of: cerebral astrocytoma, cerebellar astrocytoma, pineal gland astrocytoma, oligodendroglioma, pituitary adenoma, craniopharyngioma, sarcoma, glioblastoma, medulloblastoma, diffuse intrinsic pontine glioma, pituitary adenoma, ependymoma, medulloblastoma, neuroectoderm cancer, neuroblastoma, hypothalamic glioma, breast cancer, lung cancer, colon cancer, cervical cancer, endometrial cancer, uterine cancer, breast cancer, ovarian cancer, oesophageal cancer, basal cell carcinoma, cholangiocarcinoma, spleen cancer, pancreatic cancer, osteosarcoma, intraocular melanoma, retinoblastoma, stomach cancer, cardiac cancer, liver cancer, hypopharyngeal cancer, laryngeal cancer, cancer of the oral cavity, nasal and paranasal cancer, salivary gland cancer nasopharyngeal cancer, throat cancer, thyroid cancer, pancreatic cancer, renal cancer, prostate cancer, rectal cancer, testicular cancer, melanoma, mesothelioma, pheochromocytoma, haematological cancers, lung cancer and acute or chronic myeloid leukaemia.

5. A variant of the erythropoietin (EPO) selected from the group consisting of: EV3 variant having the amino acid sequence SEQ ID NO: 9, EV4 variant having the amino acid sequence SEQ ID NO: 10, EV1-4 variant having the amino acid sequence SEQ ID NO: 11, EV1-5 variant having the amino acid sequence SEQ ID NO: 12, EV1-1 variant having the amino acid sequence SEQ ID NO: 13, EV2-1 variant having the amino acid sequence SEQ ID NO: 14, for use as a diagnostic agent.

6. The variant for use according to claim 5, wherein said diagnostic agent is in the diagnosis of an oncological pathology selected from group consisting of brain astrocytoma, cerebellar astrocytoma, pineal gland astrocytoma, oligodendroglioma, pituitary adenoma, craniopharyngioma, sarcoma, glioblastoma, medulloblastoma, diffuse intrinsic pontine glioma, pituitary adenoma, ependymoma, medulloblastoma, neuroectodermal tumour, neuroblastoma, hypothalamic glioma, mammary tumour, pulmonary tumour, colon cancer, cervical cancer, endometrial cancer, uterine cancer, breast cancer, ovarian cancer, oesophageal cancer, basal cell cancer, cholangiocarcinoma, spleen cancer, pancreatic cancer, osteosarcoma, intraocular melanoma, retinoblastoma, stomach cancer, cardiac cancer, liver cancer, hypopharyngeal cancer, laryngeal cancer, cancer of the oral cavity, nasal and paranasal cancer, salivary gland cancer, nasopharyngeal cancer, throat cancer, thyroid cancer, pancreatic cancer, renal cancer, prostate cancer, rectal cancer, testicular cancer, melanoma, mesothelioma, pheochromocytoma, haematological cancers, lung cancer and acute or chronic myeloid leukaemia, proliferative pathologies, chronic inflammatory diseases on an autoimmune and non-autoimmune basis, neurodegenerative diseases, and in the treatment of patients undergoing organ or tissue transplantation.

7. The variant for use according to any one of claim 5 or 6, wherein said oncological pathology is drug resistant.

8. The variant for use according to any one of claims 5 to 7, wherein said diagnostic agent is for the molecular diagnosis in oncology, for prognostic purposes and for the personalisation of the therapy.

9. The variant for use according to any one of claims 5 to 8, wherein said diagnostic agent is for measuring the tissue or circulating levels of a variant of the erythropoietin (EPO).

10. The variant for use according to any one of claims 5 to 9, wherein said diagnostic agent is associated with the measurement of the expression levels of EPO, EPOR, EPHB4, CSF2RB receptors or the methylation of the promoter of the same genes.

11. A pharmaceutical composition comprising a negative functional modulator of at least one variant of the erythropoietin (EPO) selected from the group consisting of: EV3 variant having the amino acid sequence SEQ ID NO: 9, EV4 variant having the amino acid sequence SEQ ID NO: 10, EV1-4 variant having the amino acid sequence SEQ ID NO: 11, EV1-5 variant having the amino acid sequence SEQ ID NO: 12, EV1-1 variant having the amino acid sequence SEQ ID NO: 13, EV2-1 variant having the amino acid sequence SEQ ID NO: 14, and/or anti Epo receptor (EpoR, EPHB4, CSF2RB), an antisense oligonucleotide, DNA decoy, RNA decoy, a ribozyme, an antagomiR, a shRNA, LNA or siRNA, for use in the treatment of an oncological pathology, a proliferative pathology, chronic inflammatory diseases on an autoimmune and non-autoimmune basis, and in the treatment of patients undergoing organ or tissue transplantation.

12. A pharmaceutical kit comprising two or more components selected from the group consisting of:
A negative functional modulator of at least one variant of the erythropoietin (EPO), wherein said variant is selected from the group consisting of: EV3 variant having the amino acid sequence SEQ ID NO: 9, EV4 variant having the amino acid sequence SEQ ID NO: 10, EV1-4 variant having the amino acid sequence SEQ ID NO: 11, EV1-5 variant having the amino acid sequence SEQ ID NO: 12, EV1-1 variant having the amino acid sequence SEQ ID NO: 13, EV2-1 variant having the amino acid sequence SEQ ID NO: 14;
a monoclonal antibody to at least one of the at least one variant of the erythropoietin (EPO), wherein said variant is selected from the group consisting of: EV3 variant having the amino acid sequence SEQ ID NO: 9, EV4 variant having the amino acid sequence SEQ ID NO: 10, EV1-4 variant having the amino acid sequence SEQ ID NO: 11, EV1-5 variant having the amino acid sequence SEQ ID NO: 12, EV1-1 variant having the amino acid sequence SEQ ID NO: 13, EV2-1 variant having the amino acid sequence SEQ ID NO: 14, wherein said antibody is a bispecific antibody, diabody, tryabody, or tetrabody;
a polynucleotide encoding a variant of the erythropoietin (EPO), wherein said variant is selected from the group consisting of: EV3 variant having the amino acid sequence SEQ ID NO: 9, EV4 variant having the amino acid sequence SEQ ID NO: 10, EV1-4 variant having the amino acid sequence SEQ ID NO: 11, EV1-5 variant having the amino acid sequence SEQ ID NO: 12, EV1-1 variant having the amino acid sequence SEQ ID NO: 13, EV2-1 variant having the amino acid sequence SEQ ID NO: 14;
a chemotherapy drug;
an antiviral drug;
a negative functional modulator of a natural or synthetic variant of the Erythropoietin receptor (EpoR, EPHB4, CSF2RB);
a negative functional modulator of Epo receptors (EpoR EPHB4, CSF2RB);
a polynucleotide encoding Epo receptors (EpoR EPHB4, CSF2RB);
a negative functional modulator of the sphingosine-1-phosphate (S1P) signalling pathway;
an inhibitor of S1P and the metabolism thereof; or
EPO mimetics that preserve the erythropoietic function.

13. A monoclonal antibody to at least one of the variants of the erythropoietin (EPO), wherein said variant is selected from the group consisting of: EV3 variant having the amino acid sequence SEQ ID NO: 9, EV4 variant having the amino acid sequence SEQ ID NO: 10, EV1-4 variant having the amino acid sequence SEQ ID NO: 11, EV1-5 variant having the amino acid sequence SEQ ID NO: 12, EV1-1 variant having the amino acid sequence SEQ ID NO: 13, EV2-1 variant having the amino acid sequence SEQ ID NO: 14.

14. The monoclonal antibody according to claim 13, wherein said antibody is a bi-specific antibody.

15. The monoclonal antibody according to claim 13, wherein said antibody is a diabody, tryabody, or tetrabody antibody.

16. A negative functional modulator of at least one variant of the erythropoietin (EPO), wherein said variant is selected from the group consisting of: EV3 variant having the amino acid sequence SEQ ID NO: 9, EV4 variant having the amino acid sequence SEQ ID NO: 10, EV1-4 variant having the amino acid sequence SEQ ID NO: 11, EV1-5 variant having the amino acid sequence SEQ ID NO: 12, EV1-1 variant having the amino acid sequence SEQ ID NO: 13, EV2-1 variant having the amino acid sequence SEQ ID NO: 14, for use as a diagnostic agent.
